(19) United States
(12) Patent Application Publication
(10) Pub. No.: US 2015/0071989 A1
(43) Pub. Date: Mar. 12, 2015

(54) LIPOSOMES FOR HEMATOLOGICAL TUMORS

(71) Applicant: University of Tennessee Research Foundation, Knoxville, TN (US)

(72) Inventors: Nivesh K. Mittal, Memphis, TN (US); George C. Wood, Bartlett, TN (US); Leonard Lothstein, Eads, TN (US); Pavan Balabathula, Memphis, TN (US); Bivash Mandal, Memphis, TN (US)

(21) Appl. No.: 14/478,643

(22) Filed: Sep. 5, 2014

Related U.S. Application Data
(60) Provisional application No. 61/874,646, filed on Sep. 6, 2013.

(51) Int. Cl. A61K 39/44; A61K 31/704; A61K 9/127

(52) U.S. Cl. CPC A61K 39/44; A61K 9/127; A61K 31/704; A61K 2039/505
USPC 424/450; 424/133.1; 424/143.1

(57) ABSTRACT

Targeted liposomes containing chemotherapeutic compounds of Formula (I) are provided for treating hematological cancers. The targeted liposomes can be targeted to cancer cells (e.g., malignant B cells) by attachment to an antibody or antibody fragment of a suitable cell surface antigen, e.g., CD22. The targeted liposomes have higher IC₅₀s for cells not having the cell surface antigen as compared to similar non-targeted liposomes. Also provided are methods of preparing the targeted liposomes.

### RELATED APPLICATIONS

The presently disclosed subject matter claims the benefit of U.S. Provisional Patent Application Ser. No. 61/874,646, filed Sep. 6, 2013; the disclosure of which is incorporated herein by reference in its entirety.

### TECHNICAL FIELD

The presently disclosed subject matter relates to targeted liposomes for the treatment of malignancies of hematological cell populations. Also provided are methods of using the targeted liposomes and methods of preparing the targeted liposomes.

### ABBREVIATIONS

μg=microgram
μm=micron or micrometer
%=percentage
° C.=degrees Celsius
AD198=N-benzyladriamycin-14-valerate
AD445=N-benzyladriamycin-14-pivalate
CDR=complementarity determining region
CMA=carboxymethoxylamine
DLS=dynamic light scattering
DNR=duanomycin
DOX=doxorubicin
DSPE=distearyl phosphatidylethanolamine
DTPA=diethylenetriamine pentaacetic acid
DTT=dithiothreitol
EDTA=ethylenediamine tetraacetic acid
EPC=egg phosphatidylcholine
HPLC=high performance liquid chromatography
HSPC=hydrogenated soy phosphatidylcholine
IC₅₀=50% inhibitory concentration
kD=kilodalton
LUV=large unilamellar vesicle
mg=milligram
mL=milliliter
MLV=multilamellar vesicle
mM=millimolar
MTT=(3-(4,5-dimethylthiazol-2-yl)-2,5-diphenyltetrazolium bromide
mV=millivolt
nm=nanometer
PEG=poly(ethylene glycol)
PKC-δ=protein kinase C-delta
PKC-ε=protein kinase C-epsilon
PLS3=phospholipid scramblase 3
SAMSA=S-acetylmercaptosuccinic anhydride
SATA=N-succinimidyl-S-acetylthioacetate
SUV=small unilamellar vesicle
TEM=transmission electron microscopy

### BACKGROUND

Hematological tumors are malignancies of a blood cell or of a hematopoietic stem cell (i.e., a hemocytoblast) or progenitor cell thereof (e.g., myeloid or lymphoid lineage cells). Thus, hematological tumors are related to one another by virtue of their development from common progenitors, e.g., in the B-lymphocyte (i.e., B cell) or T-lymphocyte (i.e., T cell) lineage. Such malignancies include, but are not limited to, lymphoma (e.g., acute or chronic lymphoma, Hodgkin's lymphoma, non-Hodgkin's lymphoma), leukemia (e.g., acute lymphocytic leukemia), multiple myeloma, Waldenstrom's macroglobulinemia, and primary amyloidosis. More particularly, exemplary malignant diseases of B cells include, for example, acute lymphocytic leukemia (ALL), diffuse large B cell lymphoma, follicular lymphoma, mucosa-associated lymphatic tissue lymphoma (MALT), small cell lymphocytic lymphoma, mantle cell lymphoma (MCL), chronic B-lymphocytic leukemia (B-CLL), chronic myelogenous leukemia, Burkitt's lymphoma, AIDS-associated and Follicular lymphomas, B-cell lymphocytic leukemia, acute lymphoblastic leukemia, B-cell prolymphocytic leukemia, precursor B lymphoblastic leukemia, and hairy cell leukemias.

In general, leukemias are a group of neoplasms arising from the malignant transformation of hematopoietic cells. Leukemic cells proliferate primarily in the bone marrow and lymphoid tissues where they interfere with normal hematopoiesis and immunity. Ultimately, they can emigrate into the peripheral blood and infiltrate other tissues. Leukemias are classified according to the cell types primarily involved, myeloid or lymphoid, and as acute or chronic, based on the history of the disease. Lymphomas are neoplastic transformations of cells that reside primarily in the lymphoid tissues. Multiple myeloma relates to neoplastic transformation of plasma cells.

B cell malignancies are often caused by genetic abnormalities and are the most common cause of cancer in people up to the age of nineteen, accounting for 130,000 new cases of cancer in the United States every year. Therapies for treating B cell malignancies include the combination drug therapies CHOP and R-CHOP, antibody-drug conjugates (e.g., Inotuzumab ozogamicin), and biological therapies, such as monoclonal antibodies (e.g., Rituximab and Epratuzumab) and cytokines (e.g., interferon-alpha). However, the commonly used CHOP and R-CHOP therapies, which include the drugs cyclophosphamide, hydroxyduanorubicin (i.e., doxorubicin (DOX)), vincristine (i.e., ONCOVIN), and prednisone, with or without the addition of the monoclonal antibody Rituximab, can have undesirable side effects, such as cardiotoxicity from DOX, and neurotoxicity from vincristine. Furthermore, while drugs can be effective against malignant cells in the bone marrow, they can often be ineffective for treating malignant cells in the blood, which can lead to relapse and a cell population that becomes multidrug resistant.

Accordingly, there is an on-going need for additional therapeutic agents for treating hematological tumors. In particular, there is an ongoing need for additional agents for treating hematological tumors with enhanced targeting ability and/or reduced toxicity to healthy cells (e.g., healthy B cells).

### SUMMARY

In some embodiments, the presently disclosed subject matter provides a method of treating a subject having a disorder associated with a malignant hematological cell population, the method comprising administering to the subject an effective amount of a liposomal composition comprising a targeted liposome, wherein the targeted liposome comprises: (a) an outer liposomal surface attached to an antibody or antibody fragment that binds to a cell surface antigen expressed by the malignant hematological cell population, and (b) a compound of Formula (I):

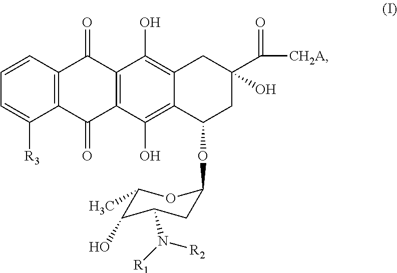

wherein: A is a C₄-C₁₀ alkanoate moiety, hemiadipate, or hemiglutarate; R₁ is H, n-propyl, or n-butyl; R₂ is benzyl, n-propyl, or n-butyl; and R₃ is H or methoxy; or a pharmaceutically acceptable salt thereof. In some embodiments, R₁ is H and R₂ is benzyl.

In some embodiments, A is a C₄-C₈ alkanoate moiety. In some embodiments, A is selected from the group consisting of:

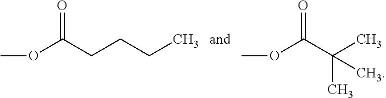

In some embodiments, the compound of Formula (I) is selected from the group consisting of:

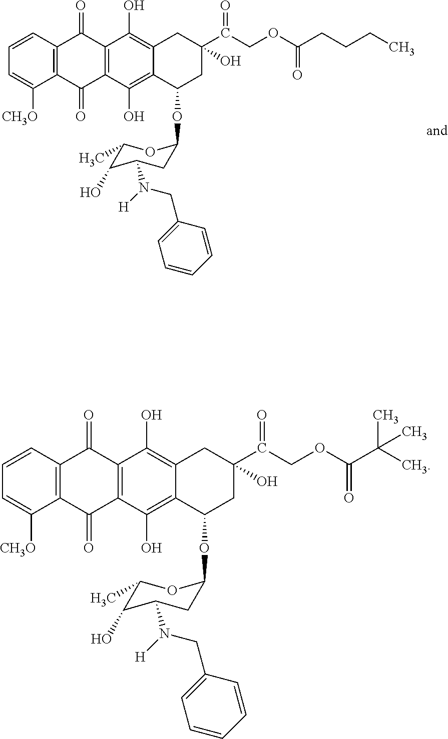

In some embodiments, the targeted antibody is a B cell- or a T cell-targeted antibody and the antibody or antibody fragment binds to a cell surface antigen expressed by a malignant B cell or a malignant T cell. In some embodiments, the antibody or antibody fragment is an anti-CD3, anti-CD4, anti-CD5, anti-CD7, anti-CD8, anti-CD10, anti-CD13, anti-CD15, anti-CD19, anti-CD20, anti-CD21, anti-CD25, anti-CD74, or anti-CD22 antibody or fragment thereof. In some embodiments, the antibody or antibody fragment is an anti-CD22 antibody or fragment thereof. In some embodiments, the antibody or antibody fragment is a monoclonal antibody, a chimeric antibody, a humanized antibody, or a fragment thereof.

In some embodiments, the disorder is selected from the group consisting of a lymphoma, a leukemia, multiple myeloma, Waldenstrom's macroglobulinemia, and primary amyloidosis. In some embodiments, the subject is a mammal.

In some embodiments, the outer liposomal surface is covalently attached to the antibody or antibody fragment via a bivalent linkage moiety. In some embodiments, the bivalent linkage moiety includes a thioether. In some embodiments, the antibody or antibody fragment is free of a free thiol moiety. In some embodiments, the bivalent linkage moiety is attached to the antibody or antibody fragment via a covalent bond to an antibody or antibody fragment amino group.

In some embodiments, the liposomal composition further comprises a polyethylene glycol chain associated with an outer liposomal surface of the targeted liposome.

In some embodiments, the targeted liposome has a mean particle size between about 80 and about 200 nm; and/or a surface charge potential of between about −5 mV and about −40 mV; and/or a drug loading of between about 1 and about 100 micrograms per milligram (μg/mg) of lipid. In some embodiments, the targeted liposome has a mean particle size between about 120 and about 140 nm. In some embodiments, the targeted liposome has a surface charge potential of between about −8 and about −12 milliVolts (mV). In some embodiments, the targeted liposome has a drug loading of about 20 μg of compound of Formula (I) to mg of lipid. In some embodiments, the targeted liposome has a circulation half-life of about 24 hours.

In some embodiments, the targeted liposome is prepared from a lipid formulation comprising hydrogenated soybean phosphatidylcholine (HSPC) and egg phosphatidylcholine (EPC). In some embodiments, the lipid formulation further comprises cholesterol, distearyl phosphatidylethanolamine-polyethylene glycol (DSPE-PEG), and distearyl phosphatidylethanolamine-polyethylene glycol-maleimide (DSPE-PEG-maleimide).

In some embodiments, the presently disclosed subject matter provides a liposomal composition comprising a targeted liposome, wherein the targeted liposome comprises: (a) an outer liposomal surface attached to an antibody or antibody fragment that binds to a cell surface antigen expressed by a malignant hematological cell population, and (b) a compound of Formula (I):

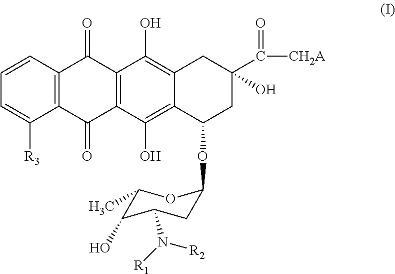

wherein: A is a C₄-C₁₀ alkanoate moiety, hemiadipate, or hemiglutarate; R₁ is H, n-propyl, or n-butyl; R₂ is benzyl, n-propyl, or n-butyl; and R₃ is H or methoxy; or a pharmaceutically acceptable salt thereof.

In some embodiments, R₁ is H and R₂ is benzyl. In some embodiments, A comprises a C₄-C₈ alkanoate. In some embodiments, A is selected from the group comprising:

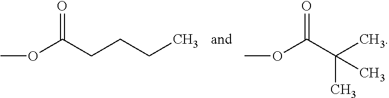

In some embodiments, the compound of Formula (I) is selected from the group comprising:

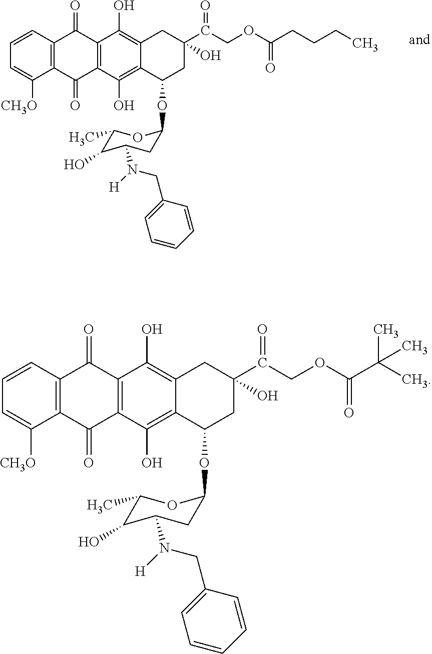

In some embodiments, the targeted liposome is a B cell- or a T cell-targeted antibody and the antibody or antibody fragment binds to a cell surface antigen expressed by a malignant B cell or a malignant T cell. In some embodiments, the composition comprises a B cell-targeted liposome, wherein the B cell-targeted liposome is a liposome having an outer surface attached to an anti-CD22 antibody or antibody fragment. In some embodiments, the liposome is covalently attached to the anti-CD22 antibody or antibody fragment via a bivalent linkage moiety. In some embodiments, the bivalent linkage moiety comprises a thioether. In some embodiments, the bivalent linkage moiety is attached to the antibody or antibody fragment via a bond to an antibody or antibody fragment amino group.

In some embodiments, the liposome further comprises a polyethylene glycol chain associated with an outer liposomal surface of the targeted liposome.

In some embodiments, the targeted liposome has a mean particle size between about 80 and about 200 nm; and/or a surface charge potential of between about −5 mV and about −40 mV; and/or a drug loading of between about 1 and about 100 micrograms per milligram (μg/mg) of lipid. In some embodiments, the targeted liposome has a meant particle size between about 120 nm and about 140 nm. In some embodiments, the targeted liposome has a surface charge potential of between about −8 mV and about −12 mV. In some embodiments, the targeted liposome has drug loading of about 20 μg of the compound of Formula (I) per mg lipid. In some embodiments, the targeted liposome has a circulation half-life of about 24 hours.

In some embodiments, the targeted liposome is prepared from a lipid formulation comprising hydrogenated soybean phosphatidylcholine (HSPC) and egg phosphatidylcholine (EPC). In some embodiments, the lipid formulation further comprises cholesterol, distearyl phosphatidylethanolamine-polyethylene glycol (DSPE-PEG), and distearyl phosphatidylethanolamine-polyethylene glycol-maleimide (DSPE-PEG-maleimide).

In some embodiments, the presently disclosed subject matter provides a method of preparing a targeted liposome, the method comprising contacting an antibody or antibody fragment that binds to a cell surface antigen expressed by a malignant hematological cell population with a derivatized liposome, wherein the derivatized liposome has an outer liposomal surface derivatized with a group that can form a bond with a group on the antibody or antibody fragment. In some embodiments, the outer liposomal surface group is a maleimide group or a haloacetyl group.

In some embodiments, the method further comprises contacting the antibody or antibody fragment with an S-acyl-containing cross-linking reagent to provide an S-acyl-derivatized antibody or antibody fragment, and contacting the S-acyl-derivatized antibody or antibody fragment with a S-acyl deblocking reagent to provide an antibody or antibody fragment having a free thiol group; prior to contacting the antibody or antibody fragment with the derivatized liposome. In some embodiments, the S-acyl-containing cross-linking reagent is N-succinimidyl-S-acetylthioacetate (SATA) or S-acetylmercaptosuccinic anhydride (SAMSA). In some embodiments, the antibody or antibody fragment is free of a free thiol moiety prior to the contacting steps.

In some embodiments, the deblocking agent is hydroxylamine or carboxymethoxylamine. In some embodiments, the deblocking agent is contacted to the S-acyl-derivatized antibody or antibody fragment in combination with a chelating agent. In some embodiments, the chelating agent is ethylenediamine tetraacetic acid (EDTA) or diethylenetriamine pentaacetic acid (DTPA).

In some embodiments, the antibody or antibody fragment is a monoclonal antibody, a chimeric antibody, a humanized antibody, or a fragment thereof. In some embodiments, the antibody of antibody fragment binds to a surface antigen expressed by a malignant B cell or T cell. In some embodiments, the antibody or antibody fragment is an anti-CD3, anti-CD4, anti-CD5, anti-CD7, anti-CD8, anti-CD10, anti-CD13, anti-CD15, anti-CD19, anti-CD20, anti-CD21, anti-CD25, anti-CD74 or anti-CD22 antibody or fragment thereof. In some embodiments, the antibody or antibody fragment is an anti-CD22 antibody or fragment thereof.

In some embodiments, the derivatized liposome comprises a compound of Formula (I).

It is an object of the presently disclosed subject matter to provide targeted (e.g., B cell and/or T cell targeted) liposomal compositions that contain therapeutic compounds, such as compounds of Formula (I), methods of using the compositions to treat malignant hematological cell populations, and methods of preparing the compositions.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1A is a schematic drawing of a targeted drug-containing liposome in accordance with an embodiment of the presently disclosed subject matter. The exemplary liposome is conjugated (via a hydrophilic polymer-derivatized lipid present as part of the liposomal lipid bilayer) to antibody and antibody fragment (i.e., Fab′ fragments) targeting agents. Hydrophilic polymer (irregular wavy lines) is also present on the outside surface of the liposome where the hydrophilic polymer is not also attached to a targeting moiety. A hydrophobic drug (indicated by the shaded ovals) is non-covalently associated with the lipid bilayer, but upon dissolution of the liposome (e.g., at physiologically relevant temperatures), the drug can be released.

FIG. 1B is a schematic drawing showing a close-up view of part of the liposome bilayer of the liposome of FIG. 1A. The dotted line on the left-hand side of the drawing indicates the inner surface of the lipid bilayer (i.e., the lipid bilayer surface on the interior of the liposome). The dotted line of the right-hand side of the drawing indicates the outer surface of the liposome. Polar head groups from the lipids of the lipid bilayer are present on both the inner and outer surface. Between the dotted lines is the hydrophobic portion of the lipid bilayer, comprising fatty acid chains from the lipids. Distributed within the fatty acid chains are hydrophobic drug molecules and cholesterol molecules.

FIG. 2 is a schematic drawing showing a synthetic scheme for conjugating Fab′ antibody fragment II to maleimide-derivatized liposome I via a free thiol group on the antibody fragment, in accordance with some embodiments of the presently disclosed subject matter.

FIG. 3 is a schematic drawing showing a synthetic scheme for conjugating antibody IV to maleimide-derivatized liposome I via an amino group on the antibody, using a sulfur (S)-acyl-containing cross-linking reagent, i.e., N-succinimidyl-S-acetylthioacetate (SATA), in accordance with some embodiments of the presently disclosed subject matter.

FIG. 4 is a flow diagram showing a method for preparing liposomes via lipid thin film hydration, in accordance with some embodiments of the presently disclosed subject matter.

FIG. 5 is a graph showing the dependence of liposomal drug loading and liposome size on the type of lipids (i.e., the ratio of hydrogenated soy phosphatidylcholine (HSPC) to egg phosphatidylcholine (EPC)) used to prepare the liposome. The striped bars (compared to the scale of the left-hand y-axis) indicate the concentration (in μg/mL) of N-benzyladriamycin-14-valerate (AD198) entrapped in the liposomes, while the heavy grey line (compared to the scale of the right-hand y-axis) indicates liposome size (in nm). The ratios on the x-axis indicate the ratio HSPC:EPC. Liposome size and drug loading concentration (“Series 1”) are also indicated below the ratios of the x-axis. Data represents the average of the measurements taken from three different liposome samples; the error bars represent±standard deviation.

FIG. 6 is a graph showing the dependence of liposomal drug loading and liposome size on the total concentration of phospholipid used to prepare the liposome. The striped bars (compared to the scale on the left-hand y-axis) indicate the concentration (in μg/mL) of N-benzyladriamycin-14-valerate (AD198) in the liposomes, while the heavy grey line (compared to the scale on the right-hand y-axis) indicates liposome size (in nm). The concentrations (in mM) of phospholipid used to prepare the liposomes were 50 mM, 75 mM, 100 mM, and 125 mM as indicated on the x-axis. Data represents the average of the measurements taken from three different liposome batches; the error bars represent±standard deviation.

FIG. 7 is a graph showing the results of a phospholipid assay after batch processing of the liposomes. The y-axis indicates the concentration (in mg/mL) of hydrogenated soy phosphatidylcholine (HSPC) present in the liposomes based on absorbance data as compared to a standard curve, while the x-axis shows the concentration (in mg/mL) of HSPC expected based on the amount present during the preparation of the liposomes. Error bars represent±standard deviation of averaged measurements.

FIG. 8 is a graph of the stability of drug loaded liposomes at 5° C. (±3° C.) over time (zero to 30 days as indicated on the x-axis). N-benzyladriamycin-14-valerate (AD198) concentration (μg/mL) in liposomes prepared using 50 mM phospholipid (ILC 50), 75 mM phospholipid (ILC 75), and 125 mM phospholipid (ILC 125) can be determined by comparing the data points shown by black circles, light grey squares, and dark grey triangles, respectively, to the left-hand y-axis. Liposome size (nm) of the ILC 50, ILC 75, and ILC 125 liposomes can be determined by comparing the data points shown by the grey “x” markings, grey “+” markings, and light grey open diamonds, respectively, to the right-hand y-axis.

FIG. 9 is a graph showing the dependence of liposomal drug loading on the theoretical concentration of drug (i.e., the concentration of drug in the drug/lipid mixture used to prepare the liposomes). The y-axis indicates the concentration (μg/mL) of N-benzyladriamycin-14-valerate (AD198) detected, while the x-axis indicates the concentration (μg/mL) of AD198 present in the drug/phospholipid mixture used to prepare the liposomes. Data represents the average of the measurements taken from three different liposome batches; the error bars represent±standard deviation.

FIG. 10 is a graph showing the dependence of liposomal surface charge potential (ζ-potential) and liposome size on the amount (1, 2, or 5 mole percentage (%)) of distearyl phosphatidylethanolamine-poly(ethylene glycol)₂₀₀₀ (DSPE-PEG₂₀₀₀) in the lipid mixture used to prepare the liposomes. Charge potential (mV) data is shown with filled circles and can be compared to the right-hand y-axis. Size data (nm) is shown in filled squares and can be compared to the left-hand y-axis. Data represents the average of the measurements taken from three different liposome batches; the error bars represent±standard deviation.

FIG. 11 is a graph showing the dependence of liposomal drug loading and liposome size on cholesterol concentration (5, 10, 15, or 30 mole %) in the lipid mixture used to prepare the liposomes. Size (nm) data is shown in the filled circles and can be compared to the right-hand y-axis, while the concentration (μg/m L) of N-benzyladriamycin-14-valerate (AD198) present in the liposomes is shown in the striped bars, which can be compared to the left-hand y-axis. Data represents the average of measurements taken from three different liposome batches; the error bars represent±standard deviation.

FIG. 12 is a graph showing drug release over time (zero to 72 hours as indicated in the x-axis) at 37° C. from liposomes prepared with different concentrations of cholesterol. The percentage (%) of drug (N-benzyladriamycin-14-valerate (AD198)) released from liposomes prepared from a lipid mixture comprising 5 mole percentage (5%) of cholesterol is shown with triangles; % drug released from liposomes prepared from a lipid mixture comprising 10 mole % (10%) cholesterol is shown with circles; and % drug released from liposomes prepared from a lipid mixture comprising 15 mole % (15%) cholesterol is shown with squares. Data represents the average of measurements taken from three different liposome batches; the error bars represent±standard deviation.

FIG. 13 is a bar graph showing the uptake of CD22 targeted, drug-loaded liposomes or of untargeted, drug-loaded liposomes in two different types of cells at 0.5, 1.5, 2.5, and 3.5 hours. The two types of cells include cells with the cell surface antigen targeted by the targeted liposomes (i.e., Daudi cells) and cells without the cell surface antigen targeted by the targeted liposomes (i.e., Jurkat cells). Fluorescence indicating liposomal uptake in the cells was measured at 533 nm. Data related to uptake of untargeted liposomes in Daudi cells is shown by the stippled bars. Data related to uptake of untargeted liposomes in Jurkat cells is shown by the black bars. Data related to uptake of CD22 targeted liposomes in Daudi cells is shown by the grey bars. Data related to uptake of CD22 targeted liposomes in Jurkat cells is shown by the open bars. The data represents the average of three measurements; the error bars represent±standard deviation.

FIG. 14A is a graph showing the in vitro cytotoxicity of targeted, drug-loaded liposomes (solid line, “CD22 Targeted NP”) and untargeted, drug-loaded liposomes (dotted line, “Untargeted NP”) in Jurkat cells after 24 hours. The Jurkat cells lack the cell surface antigen (CD22) to which the antibody of the targeted liposomes binds. For comparison, the in vitro cytotoxic effects of free (solution) drug (N-benzyladriamycin-14-valerate (AD198)) are also shown (dashed line, “Solution Drug”). Cytotoxicity is expressed as percentage (%) cell kill as measured via (3-(4,5-dimethylthiazol-2-yl)-2,5-diphenyltetrazolium bromide (MTT) cell viability assay. The data represents the average of three measurements; the error bars represent±standard deviation.

FIG. 14B is a graph showing the in vitro cytotoxicity of targeted, drug-loaded liposomes (solid line, “CD22 Targeted NP”) and untargeted, drug-loaded liposomes (dotted line, “Untargeted NP”) in Daudi cells after 24 hours. The Daudi cells have the cell surface antigen (CD22) to which the antibody of the targeted liposomes binds. For comparison, the in vitro cytotoxic effects of free (solution) drug (N-benzyladriamycin-14-valerate (AD198)) are also shown (dashed line, “Solution Drug”). Cytotoxicity is expressed as percentage (%) cell kill as measured via (3-(4,5-dimethylthiazol-2-yl)-2,5-diphenyltetrazolium bromide (MIT) cell viability assay. The data represents the average of three measurements; the error bars represent±standard deviation.

FIG. 15A is a graph showing the in vitro cytotoxicity of targeted, drug-loaded liposomes (solid line, “CD22 Targeted NP”) and untargeted, drug-loaded liposomes (dotted line, “Untargeted NP”) in Jurkat cells after 48 hours. The Jurkat cells lack the cell surface antigen (CD22) which the antibody of the targeted liposomes binds. For comparison, the in vitro cytotoxic effects of free (solution) drug (N-benzyladriamycin-14-valerate (AD198); dashed line; “Solution Drug”) and the in vitro cytotoxic effects of a blank liposome (i.e., liposome without drug; dashed and dotted line; “Blank NP”) are also shown. Cytotoxicity is expressed as percentage (%) cell kill as measured via (3-(4,5-dimethylthiazol-2-yl)-2,5-diphenyltetrazolium bromide (MTT) assay. The data represents the average of three measurements; the error bars represent±standard deviation.

FIG. 15B is a graph showing the in vitro cytotoxicity of targeted, drug-loaded liposomes (solid line, “CD22 Targeted NP”) and untargeted, drug-loaded liposomes (dotted line, “Untargeted NP”) in Daudi cells after 48 hours. The Daudi cells have the cell surface antigen (CD22) to which the antibody of the targeted liposomes binds. For comparison, the in vitro cytotoxic effects of free (solution) drug (N-benzyladriamycin-14-valerate (AD198); dashed line; “Solution Drug”) and the in vitro cytotoxic effects of a blank liposome (i.e., liposome without drug; dashed and dotted line, “Blank NP”) are also shown. Cytotoxicity is expressed as percentage (%) cell kill as measured via (3-(4,5-dimethylthiazol-2-yl)-2,5-diphenyltetrazolium bromide (MTT) cell viability assay. The data represents the average of three measurements; the error bars represent±standard deviation.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims. Thus, for example, reference to “a compound” includes mixtures of one or more compounds, two or more compounds, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Branched alkyl” refers to an alkyl group substituted with another alkyl. Exemplary “branched alkyl” groups include isopropyl, sec-butyl, iso-butyl, tert-butyl, neo-pentyl and the like.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Benzyl” refers to the aryl-alkyl-group, (C₆H₅)CH₂—. An aryl-alkyl group can also be referred to herein as “aralkyl” and can include other aryl-alkyl- or alkyl-aryl-groups, wherein the aryl and alkyl are as described above and can include one or more aryl or alkyl group substituents.

As used herein the term “alkanoate” refers to an —O—C(═O)-alkyl group. Alkanoates include, but are not limited to, pivalate, butyrate (i.e., n-butanoate), isobutanoate, valerate (i.e., n-pentanoate), isovalerate (i.e., 2-methylbutanoate), caprate (i.e., n-hexanoate), heptanoate, and caprylate (i.e., n-octanoate).

“Amino” can refer to the —NH₂ group (i.e., a primary amino group). “Alkylamino” refers to an NHR group, wherein R is an alkyl group. “Dialkylamino” refers to an amino group wherein both hydrogen atoms have been replaced by alkyl groups.

The term “thiol” or “free thiol” can refer to the —SH group. Carbon bonded thiols can also be referred to as “sulfhydryl” groups.

In some embodiments, the term “bivalent” refers to a group that can bond (e.g., covalently bond) or is bonded to two other groups, such as other alkyl, aralkyl, cycloalkyl, or aryl groups. Typically, two different sites on the bivalent group (e.g., two different atoms) can bond to groups on other molecules. For example, the bivalent group can be an alkylene group.

“Alkylene” can refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, carbonyl (—C(═O)—), sulfur or substituted or unsubstituted nitrogen atoms (also referred to herein as “alkylaminoalkyl”), wherein the nitrogen substituent is alkyl as previously described.

“Arylene” refers to a bivalent aryl group.

The term “thioether” can refer to a group having the moiety —R—S—R′—, wherein R and R′ are independently alkylene or arylene. Thus, “thioether” refers to a group having a carbon-sulfur-carbon bond sequence.

As used herein, the term “acyl” can refer to an organic carboxylic acid group wherein the —OH of the carboxylic acid group has been replaced with another substituent. Thus, an acyl group can be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Exemplary acyl groups include acetyl and benzoyl.

The term “S-acyl” refers to the —S-acyl group (i.e., —S—C(═O)R). An exemplary S-acyl group is S-acetyl (i.e., —S—C(═O)—CH₃).

The term “S-acyl deblocking agent” refers to a reagent that can react with an S-acyl group to provide a thiol. For example, such agents can include a nucleophilic group that reacts with the carbonyl carbon of the S-acyl group. Exemplary S-acyl deblocking agents include hydroxylamine and carboxymethoxylamine.

The term “cross-linking reagent” refers to a compound that includes at least two reactive functional groups (or groups that can be deblocked or deprotected to provide reactive functional groups). In some embodiments, the two reactive functional groups have different chemical reactivity (e.g., the two reactive functional groups are reactive (e.g., form bonds, such as covalent bonds) with different types of functional groups on other molecules, or one of the two reactive functional groups tends to react more quickly with a particular functional group on another molecule than the other reactive functional group). Thus, the cross-linking reagent can be used to link (e.g., covalently) two other entities (e.g., molecules, proteins, nucleic acids, liposomes, nanoparticles, microparticles, etc.) to form a conjugate. The term “immunoconjugate” refers to a conjugate between an antibody (or antibody fragment) and another molecule or entity (e.g., a liposome or other nanoparticle).

As used herein the term “apoptosis” refers to programmed cell death, a cellular process comprising the self-destruction of a cell in a multicellular organism.

The term “cancer” as used herein refers to diseases caused by uncontrolled cell division and the ability of cells to metastasize, or to establish new growth in additional sites. The terms “malignant”, “malignancy”, “neoplasm”, “tumor” and variations thereof refer to cancerous cells or groups of cancerous cells. In some embodiments, the presently disclosed methods and compositions can be used to treat neoplasms. In some embodiments, the presently disclosed methods and compositions can cause apoptosis of malignant cells.

In some embodiments, the term “disorder associated with a malignant hematological cell population” refers to a disorders (e.g., a blood disorder) characterized by a neoplasm of a blood cell or a hematopoietic stem cell or progenitor cell thereof (e.g., myeloid or lymphoid lineage cells). Thus, the disorders can be associated with neoplasms of, for example, B-lymphocyte or T-lymphocyte lineage cells. Such disorders can include, but are not limited to, lymphoma (e.g., acute or chronic lymphoma, Hodgkin's lymphoma, non-Hodgkin's lymphoma), leukemia (e.g., acute lymphocytic leukemia), multiple myeloma, Waldenstrom's macroglobulinemia, and primary amyloidosis. Exemplary malignant diseases of B cells include, for example, acute lymphocytic leukemia (ALL), diffuse large B cell lymphoma, follicular lymphoma, mucosa-associated lymphatic tissue lymphoma (MALT), small cell lymphocytic lymphoma, mantle cell lymphoma (MCL), chronic B-lymphocytic leukemia (B-CLL), chronic myelogenous leukemia, Burkitt's lymphoma, AIDS-associated and Follicular lymphomas, B-cell lymphocytic leukemia, acute lymphoblastic leukemia, B-cell prolymphocytic leukemia, precursor B lymphoblastic leukemia, and hairy cell leukemias. In some embodiments, the disorder is drug resistant.

The term “drug-resistant” can refer to a cancer which never responded to a chemotherapeutic drug or which initially responded to an anti-cancer drug (e.g., DOX or DNR), but which has become resistant to the anti-cancer drug (i.e., the anti-cancer drug is no longer effective in treating the cancer). Cancers that have or have developed resistance to two or more anti-cancer drugs are said to be “multi-drug resistant”. For example, it is common for cancers to become resistant to three or more anti-cancer agents, often five or more anti-cancer agents and at times ten or more anti-cancer agents. The term “refractory” also can be used to describe a cancer that has become resistant to a previously effective treatment.

“Cardioprotective” as used herein can refer to compounds and/or methods that reduce or prevent damage to the heart, or more particularly, to heart muscle cells. The heart damage prevented by use of cardioprotective compounds and/or methods can be acute damage or chronic damage. Thus, “cardioprotective” can relate to, in some embodiments, preventing or reducing congestive heart failure, cardiac ischemic, hypertension, hypotension, arrhythmias, and/or cardiomyopathy. Damage to the heart can be manifested through one or more parameters or indicia including changes to stroke volume, ejection fraction, end diastolic fraction, stroke work, arterial elastance, or by an increase in heart weight to body weight ratio. Thus, cardioprotection can be assessed by determining a lack of (or minimal) changes to one or more of these parameters and/or to reduced changes as compared to the changes that would occur with the use of other methods and/or compounds (e.g., other chemotherapeutic compounds). As used herein, cardioprotection also can be used to refer to the reduction or inhibition of apoptosis in cardiomyocytes.

The term “liposome” refers to an artificially created vesicle comprising one or more concentric lipid bilayers. In some embodiments, the liposome can comprise one or more amphipathic lipid, having hydrophobic and polar head group moieties. Examples of lipids include, but are not limited to, phospholipids and glycolipids, such as phosphatidylcholine, phosphatidylethanolamine, phosphatidic acid, phosphatidylglycerol, phosphatidylinositol, and sphingomyelin. The lipid bilayers employed can be of any conventional composition and can either contain cholesterol or can be cholesterol-free.

The term “outer liposomal surface” refers to an outer surface of the liposome as a whole (e.g, the outer surface of the outermost lipid bilayer).

“Antibodies” refer to polypeptides substantially encoded by an immunoglobulin gene or immunoglobulin genes, or fragments thereof, which specifically bind and recognize an antigen. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Thus, the term “antibody” as used herein, can refer to an immunoglobulin molecule, which is able to specifically bind to a particular epitope on an antigen. As used herein, an antibody is intended to refer broadly to any immunologic binding agent such as IgG, IgM, IgA, IgD and IgE. Antibodies can be intact immunoglobulins derived from natural sources or from recombinant sources and can be immunoactive portions of intact immunoglobulins. The antibodies can exist in a variety of forms including, for example, polyclonal antibodies, monoclonal antibodies, Fv, Fab and F(ab)₂ fragments, as well as single chain antibodies, chimeric antibodies, and humanized antibodies.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

An “antibody fragment” is a portion of an antibody such as F(ab′)₂, F(ab)₂, Fab′, Fab, Fv, scFv, single domain antibodies (DABs or VHHs) and the like. Regardless of structure, an antibody fragment in accordance with the presently disclosed subject matter binds with the same antigen that is recognized by the intact antibody. For example, an anti-CD22 antibody fragment binds with an epitope of CD22. Thus, the term “antibody fragment” includes isolated fragments comprising the variable regions, such as the “Fv” fragments comprising the variable regions of the heavy and light chains, recombinant single chain polypeptide molecules in which light and heavy chain variable regions are connected by a peptide linker (“scFv proteins”), and minimal recognition units comprising the amino acid residues that mimic the hypervariable region.

Antibody fragments can be produced by digestion with various peptidases. For example, pepsin digests an antibody below the disulfide linkages in the hinge region to produce F(ab)′₂, a dimer of Fab which itself is a light chain joined to V_{H}-C_{H1} by a disulfide bond. The F(ab)′₂ can be reduced under mild conditions to break the disulfide linkage in the hinge region, thereby converting the F(ab)′₂ dimer into a Fab′ monomer. The Fab′ monomer is essentially a Fab with part of the hinge region. While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An antibody, is “reactive with” or “binds to” an antigen if it interacts with the antigen. The term “binding specificity,” “specifically binds to an antibody” or “specifically immunoreactive with,” when referring to an antigen (e.g., a protein, peptide or carbohydrate), refers to a binding reaction which is determinative of the presence of the antigen in the presence of a heterogeneous population of proteins and other biologics. Thus, under designated immunoassay conditions, the specified antibodies bind to a particular antigen and do not bind in a significant amount to other proteins or carbohydrates present in the sample. Specific binding to an antibody under such conditions can require an antibody selected for its specificity towards a particular antigen. For example, antibodies raised to the CD22 antigen can be selected to provide antibodies that are specifically immunoreactive with CD22 protein and not with other proteins. A variety of immunoassay formats can be used to select antibodies specifically immunoreactive with a particular protein or carbohydrate. For example, solid-phase ELISA immunoassays are routinely used to select antibodies specifically immunoreactive with a particular protein or carbohydrate.

A “chimeric antibody” is a recombinant protein that contains the variable domains including the complementarity determining regions (CDRs) of an antibody derived from one species, such as a rodent antibody, while the constant domains of the antibody molecule are derived from those of a human antibody. For veterinary applications, the constant domains of the chimeric antibody can be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains. The constant domains of the antibody molecule are derived from those of a human antibody.

Thus, the term “humanized” can refer to an antibody wherein the constant regions have at least about 80% or greater homology to human immunoglobulin. Additionally, some of the nonhuman, such as murine, variable region amino acid residues can be modified to contain amino acid residues of human origin.

A “human antibody” can be an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. A fully human antibody also can be constructed by genetic or chromosomal transfection methods, as well as by phage display technology, all of which are known in the art. Human antibodies can also be generated by in vitro activated B cells.

As used herein, the term “antibody fusion protein” is a recombinantly produced antigen-binding molecule in which an antibody or antibody fragment is linked to another protein or peptide, such as the same or different antibody or antibody fragment or a peptide. The fusion protein can comprise a single antibody component, a multivalent or multispecific combination of different antibody components or multiple copies of the same antibody component. The fusion protein can additionally comprise an antibody or an antibody fragment and a therapeutic agent. Examples of therapeutic agents suitable for such fusion proteins include immunomodulators and toxins.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “bispecific antibody” is an antibody that can bind simultaneously to two targets which are of different structure. Bispecific antibodies (bsAb) and bispecific antibody fragments (bsFab) can have at least one arm that specifically binds to, for example, a B-cell. A variety of bispecific antibodies can be produced using molecular engineering.

The terms “surface marker” or “surface antigen” refer to a protein, carbohydrate, or glycoprotein present on the surface of a cell. Different types of cells express different cell surface markers and therefore cells can be identified by the presence of a cell surface marker. For example, malignant B cells can overexpress CD22. Thus, the binding of an antibody that recognizes CD22 can identify that cell as a B cell. CD74, CD19, and CD20 are other examples of cell surface markers that can be found on B cells. CD3, CD4, and CD8 are examples of cell surface markers that can be found on T cells.

### II. GENERAL CONSIDERATIONS

Liposomes are micro- or nanoscopic particles that are made up of one or more lipid bilayers enclosing an internal compartment. Liposomes can be categorized into multilamellar vesicles, multivesicular liposomes, unilamellar vesicles and giant liposomes. Liposomes have been widely used as carriers for a variety of agents such as drugs, cosmetics, diagnostic reagents, and genetic material. Since liposomes comprise non-toxic lipids, they generally have low toxicity and therefore are useful in a variety of pharmaceutical applications. In particular, liposomes can be useful for increasing the circulation lifetime of agents that have a short half-life in the bloodstream. In addition, liposome encapsulated drugs often have biodistributions and toxicities which differ from those of free drug. For in vivo delivery, the sizes, charges, and surface properties of the liposomes can be tailored by varying the preparation methods and/or adjusting the lipid makeup of the lipid bilayer(s). More particular targeting moieties can also be attached to the outer surface of the liposomes.

In some embodiments, the presently disclosed subject matter provides a liposomal composition (e.g., a liposomal suspension) for treating disease, such as cancer. In particular, the liposomal composition can be used to treat hematological cancers (e.g., cancers related to blood cells, such as white blood cells) and/or cells derived from the bone marrow (e.g., hematopoietic stem cells and their progenitor cells, such as myeloid and lymphoid cells). Thus, the presently disclosed compositions can include a liposome and a therapeutic agent (e.g., a chemotherapeutic agent). In some embodiments, the therapeutic agent is entrapped within the liposome. When the therapeutic agent is an aqueous-soluble compound or salt, using conventional liposome technology, the same can be incorporated into lipid vesicles. In such an instance, due to the water solubility of the therapeutic agent, the therapeutic agent can be substantially entrained or entrapped within the hydrophilic center or core of the liposomes. When the therapeutic agent is water-insoluble, again employing conventional liposome formation technology, the therapeutic agent can be substantially entrained or entrapped within the hydrophobic lipid bilayer that forms the structure of the liposome. In either instance, the liposomes that are produced can be reduced in size, as through the use of standard sonication and homogenization techniques.

In some embodiments, the presently disclosed liposomal composition can be a targeted liposomal composition, wherein the liposome is attached to a targeting moiety that targets the liposome for delivery to a cell population of interest (e.g., a neoplasmic B cell population). In some embodiments, the targeting moiety is an antibody or antibody fragment that preferentially or particularly binds a cell surface antigen on the cells of interest. Cell surface antigens associated with malignant hematological cells include, but are not limited to, CD3, CD4, CD5, CD7, CD8, CD10, CD13, CD15, CD19, CD20, CD21, CD25, CD74, and CD22. The antibody or antibody fragment can be associated with an outer liposomal surface of the liposomes, e.g., by covalent attachment to the outer liposomal surface, such as via a bivalent linkage moiety attached to one of the lipid components of the liposome lipid bilayer.

In some embodiments, the outer liposomal surface is associated with (e.g., covalently attached to or non-covalently coated with) a group that can modulate (e.g., increase) the circulation half-life of the liposomes. Such groups can include polymers, such as, but not limited to, polyethylene glycol (PEG) chains and other hydrophilic polymers (e.g., polyvinylpyrrolidone, polymethylacrylamide, polylactic acid, polyglycolic acid, celluloses, etc.). When PEG chains are used, for instance, they can have molecular weights of between about 500 and about 10,000 daltons (e.g., 500; 1,000; 2,000; 5,000; or about 10,000 daltons). In some embodiments, the PEG chain can have a molecular weight of about 2,000 daltons.

Accordingly, in some embodiments, the presently disclosed subject matter provides a targeted liposome comprising: (a) an outer liposomal surface attached to an antibody and/or antibody fragment that binds to a cell surface antigen expressed by a malignant hematological cell population, and (b) a chemotherapeutic agent. In some embodiments, the chemotherapeutic agent is a compound of Formula (I):

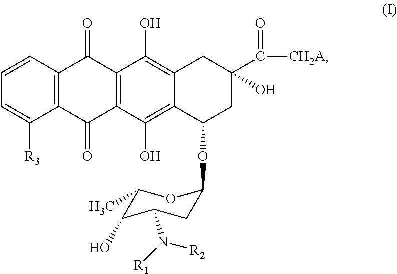

wherein: A comprises a C₄-C₁₀ alkanoate moiety, hemiadipate, or hemiglutarate; R₁ is H, n-propyl, or n-butyl; R₂ is benzyl, n-propyl, or n-butyl; and R₃ is H or methoxy; or a pharmaceutically acceptable salt thereof.

In some embodiments, the compound of Formula (I) is a compound wherein R₁ and R₂ are n-propyl or n-butyl, or wherein R₁ is H, n-propyl, or n-butyl and R₂ is benzyl.

In some embodiments, the compound of Formula (I) is a compound wherein R₁ is H and R₂ is benzyl. In some embodiments, A is a C₄-C₈ alkanoate. In some embodiments, A is selected from the group comprising:

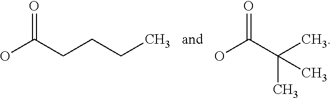

In some embodiments, the compound of Formula (I) is selected from the group comprising N-benzyladriamycin-14-valerate (AD198) and N-benzyladriamycin-14-pivalate (AD445), represented respectively by the structures:

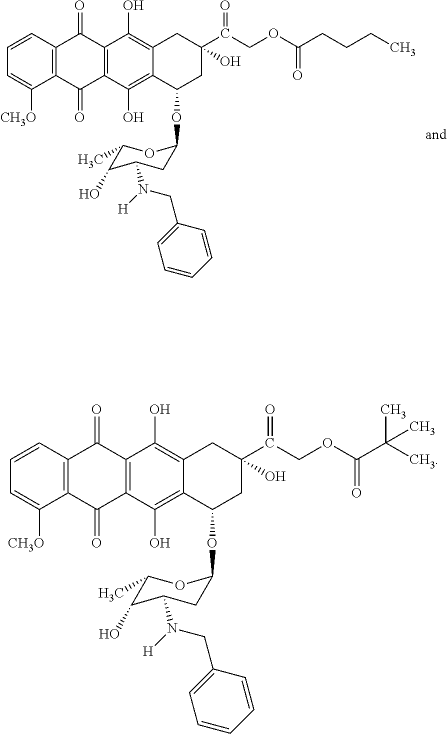

For additional information regarding N-alkyl and N-aralkyl adriamycin derivatives, see U.S. Pat. No. 4,610,977 to Israel et al. and U.S. Pat. No. 7,541,341 to Lothstein et al., which are incorporated herein by reference in their entireties.

In some embodiments, the compound of Formula (I) is a compound with enhanced antitumor activity and/or lower toxicity compared to adriamycin (also known as doxorubicin (DOX)) and/or duanomycin (DNR). In some embodiments (e.g., when R₂ is benzyl and R₁ is H), the biochemical mechanism(s) of action and pharmacological effects of the presently disclosed compounds, in vitro and in vivo, can differ significantly from those of other structurally-related anthracycline anti-tumor drugs, such as DNR and DOX. Without wishing to be bound to any one theory, it is believed that DNR and DOX concentrate in the nuclei of cells, where they bind strongly with DNA and interfere with the action of the enzyme DNA topoisomerase II (topo II), whereas, in some embodiments, the compounds of Formula (I) are believed to not enter the nucleus, bind minimally to isolated DNA, and have no effect on topo II at cytotoxic doses. In some embodiments, the administering of an effective amount of a compound of Formula (I) can induce the translocation of protein kinase C-delta (PKC-δ) to the mitochondria of a cancer cell to induce the phosphorylation of phospholipid scramblase 3 (PLS3), thereby inducing apoptosis in the cancer cell. The compounds of Formula (I) can also be cardioprotective. The cardioprotection can occur through the translocation and activation of protein kinase C-epsilon (PKC-δ), which, in turn, can activate a mechanism in cardiac myocytes that inhibits apoptosis through protection of mitochondria.

In some embodiments, the physical characteristics of the targeted liposomes can be tailored to provide the liposomes with a circulation half-life that allows for the most effective treatment of non-solid tumors and/or cancer cells that are mobile within the subject. In some embodiments, the liposomes can have a circulation half-life of between about 8 hours and about 48 hours (i.e., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 37, 38, 39, 40, 41, 42, 43, 44, 45, 46, 47, or 48 hours). In some embodiments, the liposomes can have a circulation half-life of between about 12 and about 24 hours. In some embodiments, the liposomes have a circulation half-life of about 24 hours.

In some embodiments, the targeted liposomes can have a mean particle size between about 80 nm and about 200 nm (i.e., about 80, 90, 100, 110, 120, 130, 140, 150, 160, 170, 180, 190, or about 200 nm). In some embodiments, the targeted liposomes can have a mean particle size between about 120 nm and about 140 nm.

In some embodiments, the targeted liposomes can have a surface charge potential (zeta (ζ)-potential) between about −5 and about −40 mV (i.e., about −5, −10, −15, −20, −25, −30, −35, or −40 mV). In some embodiments, the targeted liposomes can have a surface charge potential of between about −8 mV and about −12 mV (e.g., about −8, −8.5, −9, −9.5, −10, −10.5, −11, −11.5 or −12 mV).

The size, surface charge potential, drug loading, and other properties of the liposomes can be tailored based on the lipid composition used to prepare the liposomes. For example, size, surface charge potential, and drug loading can be adjusted by adjusting the ratio of egg phosphatidylcholine (EPC) and hydrogenated soy phosphatidylcholine (HSPC) used to prepare the liposomes. Additional components (e.g., cholesterol, distearyl phosphatidylethanolamine (DSPE)-PEG (e.g., DSPE-PEG₂₀₀₀), and DSPE-PEG-maleimide (e.g., DSPE-PEG₂₀₀₀-maleimide)) can be added to further adjust one or more of the size, charge potential, circulation half-life, and the number of targeting groups that can be attached to the liposome.

In some embodiments, the lipid formulation used to prepare the liposomes can comprise EPC and HSPC as the major lipid components and between about 5 and about 30 mole % (e.g., about 5, 10, 15, 20, 25 or 30 mole %) cholesterol, between about 0.5 and about 1 mole % (e.g., about 0.5, 0.6, 0.7, 0.8, 0.9, or 1.0 mole %) DSPE-PEG₂₀₀₀, and between about 0.5 and about 5 mole % (e.g., about 0.5, 1.0, 1.5, 2.0, 2.5, 3.0, 3.5, 4.0, 4.5, or 5.0 mole %) DSPE-PEG₂₀₀₀-maleimide. In some embodiments, the lipid formulation used to prepare the liposomes comprises between about 5 and about 15 mole % cholesterol (e.g., about 5, 6, 7, 8, 9, 10, 11, 12, 13, 14, or 15 mole % cholesterol). While maleimide-derivatized lipids are provided herein as exemplary derivatized lipids that can be incorporated into the lipid formulation to provide for an outer liposomal surface group capable of conjugating with, for instance, targeting moieties (e.g., antibodies), lipids derivatized with other reactive groups typically used in conjugation chemistries can also be used, as would be understood by one of skill in the art.

In some embodiments, the targeted liposomes can have drug loading of about 1 to about 100 micrograms (e.g., about 1, 5, 10, 20, 30, 40, 50, 60, 70, 80, 90, or 100 micrograms (μg)) of the compound of Formula (I) per mg of lipid. In some embodiments, the targeted liposomes can have a drug loading of about 20 μg of the compound of Formula (I) per mg of lipid.

In some embodiments, the targeted liposomes are B cell- or T cell-targeted liposomes and the liposomes are attached to an antibody or antibody fragment that binds to a cell surface antigen expressed by a malignant B cell or a malignant T cell. For instance, the antibody or antibody fragment can bind to a cell surface antigen selected from the group including, but not limited to, CD3, CD4, CD7, CD8, CD10, CD13, CD15, CD19, CD20, CD21, CD25, CD74 and CD22. In some embodiments, the antibody is a monoclonal antibody, a chimeric antibody or a humanized antibody. In some embodiments, the antibody fragment is derived from a monoclonal antibody, a chimeric antibody or a humanized antibody.

Suitable antibodies or antibody fragments can be prepared via methods known in the art (e.g., via elicitation in a mammal using suitable immunogens). For example, a variety of methods for producing monoclonal antibodies are known in the art. A monoclonal antibody directed against or reactive with B cells or T cells (e.g., human B cells or human T cells) can be obtained by using an immunogen or combinations of immunogens to immunize mice and screening hybridoma supernatant against cells which express the desired antigen or by a screening assay designed to be specific for monoclonal antibodies directed against the antigen of interest. Useful cell lines for screening for the antibodies against B cells include the Burkitt's lymphoma cell lines Daudi, Calif.-46, and Raji. Alternatively, suitable antibodies or antibody fragments directed against B cells or T cells can be obtained commercially from suppliers of immunological reagents.

In some embodiments, the antibody or antibody fragment is an anti-CD22 antibody or antibody fragment. In some embodiments, the antibody or antibody fragment is a monoclonal anti-CD22 antibody (e.g., Pierce catalog number MA1-7635, Pierce Antibodies, Thermo Fisher Scientific, Rockford, Ill., United States of America). This antibody does not comprise a free thiol group. Thus, the antibody can be derivatized with a suitable reagent for introducing a thiol (e.g., an S-acyl cross-linking reagent) if desired. However, any other suitable conjugation chemistry can be used for attaching the antibody or antibody fragment to the liposome, as described hereinbelow. In some embodiments, the antibody fragment is a Fab′ fragment. The Fab′ fragment can be conjugated to the liposome using a free thiol on the Fab′ fragment.

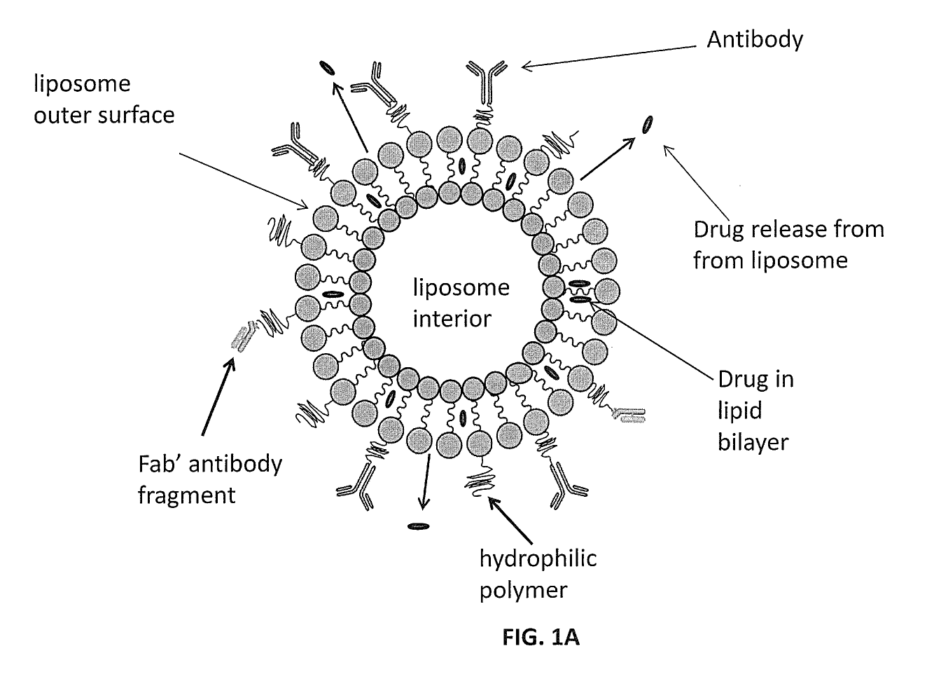

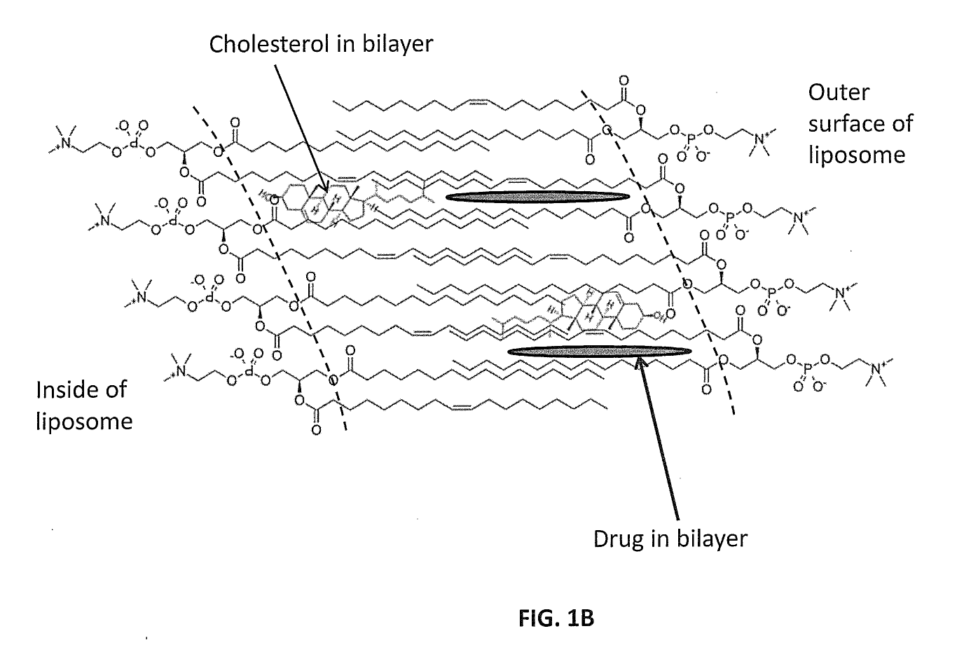

FIG. 1A shows an exemplary liposome of the presently disclosed subject matter. As shown in FIG. 1A, the liposome can be unilamellar (i.e., have a single lipid bilayer). The lipid bilayer is represented by the two concentric circles of shaded circles separated by regular wavy lines. The drug compound (e.g., the compound of Formula (I) or other hydrophobic therapeutic agent) represented by the shaded oval, is entrapped in the lipid bilayer (e.g., non-covalently associated with the lipid fatty acid chains). If a supplementary hydrophilic drug is included as part of the liposomal composition, it can be entrapped in the hydrophilic liposome interior. Under appropriate conditions, the drug(s) can be released from the liposome. The outer surface of the liposome can have one or more covalently or non-covalently attached targeting moieties, e.g., intact antibodies and/or Fab′ fragments. Referring again to FIG. 1A, targeting moieties (whole antibodies and Fab′ fragments) are covalently attached to the outer liposomal surface via hydrophilic polymer-derivatized lipids that make up the lipid bilayer, although it is also possible for the targeting moieties to be non-covalently associated with the outer surface of the liposome, e.g., via non-covalent interactions with suitably derivatized lipids. In FIG. 1A, hydrophilic polymers are represented by the irregular wavy lines. The covalent attachment of the targeting moiety to the liposome can further include a linking moiety between the targeting moiety and the hydrophilic polymer and/or between the hydrophilic polymer and the lipid. Additionally, the liposome outer surface can also have attached hydrophilic polymers (e.g., PEG₂₀₀₀) where the hydrophilic polymer is not further attached to a targeting moiety. The hydrophilic polymers can extend the circulation half-life of the liposome.

FIG. 1B shows a closer view of a section of the lipid bilayer. To the left-hand side (i.e., on the left side of the dotted line on the left) are the polar head groups of the lipids that are in the interior (i.e., the hydrophilic core) of the liposome. To the right-hand side (i.e., on the right side of the dotted line on the right) are the polar head groups of the lipids that are on the outer surface of the liposome. In between the dotted lines is the hydrophobic portion of the bilayer containing the fatty acid chains of the lipids making up the lipid bilayer. A hydrophobic drug compound (shaded oval), such as a compound of Formula (I), is associated with the fatty acid chains of the bilayer via non-covalent associations. Also associated with the fatty acid chains are cholesterol molecules.

### III. METHODS OF TREATING DISORDERS ASSOCIATED WITH MALIGNANT HEMATOLOGICAL CELL POPULATIONS

In some embodiments, the presently disclosed subject matter provides a method of treating a subject having a disorder associated with a malignant hematological cell population. In some embodiments, the method comprises administering to the subject an effective amount of a liposomal composition comprising a targeted liposome. The targeted liposome can comprise a suitable targeting moiety and a chemotherapeutic agent.

In some embodiments, the targeted liposome can comprise an outer liposomal surface attached to an antibody or antibody fragment that binds to a cell surface antigen expressed by a malignant hematological cell population. In some embodiments, the chemotherapeutic agent is a compound of Formula (I):

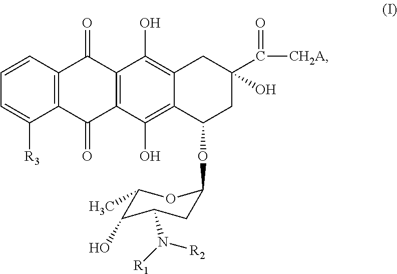

wherein: A comprises a C₄-C₁₀ alkanoate moiety, hemiadipate, or hemiglutarate; R₁ is H, n-propyl, or n-butyl; R₂ is benzyl, n-propyl, or n-butyl; and R₃ is H or methoxy; or a pharmaceutically acceptable salt thereof.

In some embodiments, the compound of Formula (I) is a compound wherein R₁ and R₂ are n-propyl or n-butyl, or wherein R₁ is H, n-propyl, or n-butyl and R₂ is benzyl.

In some embodiments, the compound of Formula (I) is a compound wherein R₁ is H and R₂ is benzyl. In some embodiments, A is a C₄-C₅ alkanoate. In some embodiments, A is selected from the group comprising:

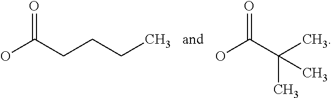

In some embodiments, the compound of Formula (I) is:

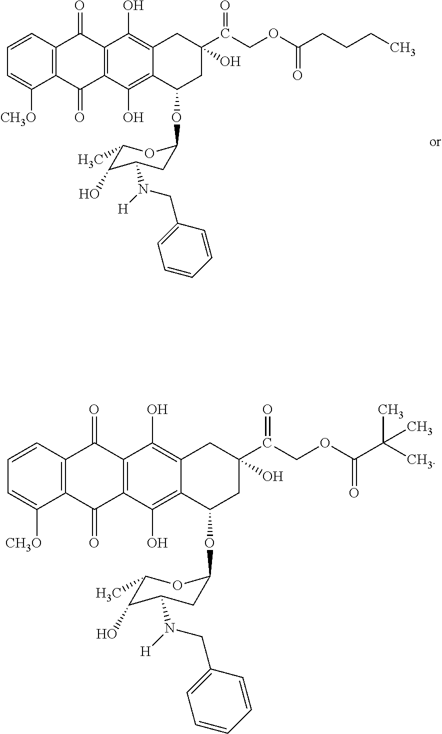

In some embodiments, the presently disclosed targeted liposomes can be used to treat drug-resistant B cell or T cell malignancies in a subject in need thereof by providing a cytoplasmically-active anthracycline derivative of Formula (I) to produce activation of protein kinase C holoenzymes to achieve the desired pharmacological effect(s). In some embodiments, cardioprotection is also provided.

In some embodiments, the targeting moiety is a B cell- or T cell-targeted antibody or fragment thereof. B cell- and T cell-targeted antibodies and antibody fragments can bind to cell surface antigens expressed by a malignant B cell or a malignant T cell. Suitable B cell- and T cell-targeted antibodies and antibody fragments include, but are not limited to, anti-CD3, anti-CD4, anti-CD5, anti-CD7, anti-CD8, anti-CD10, anti-CD13, anti-CD15, anti-CD19, anti-CD20, anti-CD21, anti-CD25, anti-CD74, or anti-CD22 antibodies or fragments thereof. In some embodiments, the targeting moiety is an anti-CD22 antibody or fragment thereof.

In some embodiments, the disorder associated with a malignant hematological cell population is a lymphoma, a leukemia, multiple myeloma, Waldenstrom's macroglobulinemia, or primary amyloidosis. In some embodiments, the disorder is selected from the group comprising, but not limited to, Hodgkin's lymphoma, non-Hodgkin's lymphoma, acute lymphocytic leukemia (ALL), diffuse large B cell lymphoma, follicular lymphoma, mucosa-associated lymphatic tissue lymphoma (MALT), small cell lymphocytic lymphoma, mantle cell lymphoma (MCL), chronic B-lymphocytic leukemia (B-CLL), chronic myelogenous leukemia, Burkitt's lymphoma, AIDS-associated and Follicular lymphoma, B-cell lymphocytic leukemia, acute lymphoblastic leukemia, B-cell prolymphocytic leukemia, precursor B lymphoblastic leukemia, and hairy cell leukemia. In some embodiments, the disorder is drug resistant (e.g., multi-drug resistant).

The subject treated in the presently disclosed subject matter in its many embodiments is desirably a human subject, although it is to be understood the methods described herein are effective with respect to all vertebrate species, which are intended to be included in the term “subject.” In some embodiments, the subject is a warm-blooded vertebrate.

More particularly, provided herein is the treatment of mammals, such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided herein is the treatment of birds, including the treatment of those kinds of birds that are endangered, kept in zoos or as pets, as well as fowl, and more particularly domesticated fowl, i.e., poultry, such as turkeys, chickens, ducks, geese, guinea fowl, and the like, as they also are of economic importance to humans. Thus, embodiments of the methods described herein include the treatment of livestock, including, but not limited to, domesticated swine (pigs and hogs), ruminants, horses, poultry, and the like. In some embodiments, the subject is a human or a dog.

Liposomes may be formulated for delivery as part of a pharmaceutical composition that includes a pharmaceutically acceptable carrier. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the liposomal pharmaceutical compositions. The presently disclosed liposomal formulations can be lyophilized to produce a lyophilizate, which can be reconstituted with a pharmaceutically acceptable carrier, such as water, to regenerate a liposomal suspension.

In some embodiments, the presently disclosed compounds of Formula (I) and/or supplementary active compounds can be employed in the liposomal compositions as pharmaceutically acceptable salts. Such pharmaceutically acceptable salts include the gluconate, lactate, acetate, tartarate, citrate, phosphate, maleate, borate, nitrate, sulfate, and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. In some embodiments, the hydrochloride salt of the presently disclosed amine-containing anthracycline compounds is made by passing hydrogen chloride gas into an anhydrous solution of the free base. Accordingly, in some embodiments, the pharmaceutically acceptable salt is a hydrochloride salt.

A pharmaceutical composition can be formulated to be compatible with its intended route of administration. Examples of routes of administration include parenteral, e.g., intravenous, intradermal, subcutaneous, oral (e.g., inhalation), transdermal (topical), transmucosal, nasal, optical, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions or dispersions and sterile powders for the extemporaneous preparation of sterile injectable solutions or dispersion. For intravenous administration, suitable carriers include, but are not limited to, physiological saline, bacteriostatic water, PEGylated Castor oil, or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, other fluids configured to preserve the integrity of the liposome, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols such as mannitol, sorbitol, and sodium chloride sometimes are included in the composition. Prolonged absorption of the injectable compositions can be brought about by including in the composition an agent which delays absorption, for example, aluminum monostearate and gelatin. Sterile injectable solutions can be prepared by incorporating the liposomes in the required amount in an appropriate solvent with one or a combination of ingredients enumerated above, as required, followed by filtered sterilization.

For administration by inhalation, the liposomes can be delivered in the form of an aerosol spray from pressured container or dispenser that contains a suitable propellant, e.g., a gas such as carbon dioxide, or a nebulizer.

Systemic administration can also be by transmucosal or transdermal means, including nasal and optical. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Delivery vehicles can also be prepared in the form of suppositories (e.g., with conventional suppository bases such as cocoa butter and other glycerides) or retention enemas for rectal delivery.

In some embodiments oral or parenteral compositions are formulated in a dosage unit form for ease of administration and uniformity of dosage. Dosage unit form as used herein refers to physically discrete units suited as unitary dosages for the subject to be treated; each unit containing a predetermined quantity of active compound and/or liposomal composition calculated to produce the desired therapeutic effect in association with the required pharmaceutical carrier.

Toxicity and therapeutic efficacy of the presently disclosed liposomes can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In some embodiments, liposomal delivery can increase the therapeutic index of the compound of Formula (I) (e.g., by increasing LD₅₀).

The data obtained from the cell culture assays and animal studies can be used in formulating a range of dosage for use in humans or another species. The dosage can lie within a range of circulating concentrations that include the ED₅₀ with little or no toxicity. The dosage can vary within this range depending upon the dosage form employed and the route of administration utilized. The therapeutically effective dose can be estimated initially from cell culture assays. A dose may be formulated in animal models to achieve a circulating plasma concentration range that includes the IC₅₀ (i.e., the concentration of the liposome which achieves a half-maximal inhibition of symptoms) as determined in cell culture. Such information can be used to more accurately determine useful doses in humans.

In some embodiments, the presently disclosed targeted liposomes provide for the treatment of a malignant hematological cell population with increased efficacy and/or reduced toxicity to healthy cells compared to methods wherein a compound of Formula (I) is administered in the absence of a targeted liposome. For example, systemic administration of AD 198 can be associated with dose related toxicities, such as neutropenia and thrombocytopenia. These toxicities can be limited by using the presently disclosed targeted liposomal compositions. In some embodiments, the use of a targeted liposome can, for example, allow for effective treatment of the subject using a lower dosage of the compound of Formula (I). In some embodiments, a dosage of lower than 1 mg/kg can be used according to the presently disclosed methods.

Targeted liposomes of the presently disclosed subject matter can be tested or used in vivo in various mouse xenograft models that are known in the art. In addition, the liposomes can be tested against canine lymphomas, which are believed to be similar to human non-Hodgkin's lymphomas.

### IV. METHODS OF PREPARING TARGETED LIPOSOMES

In some embodiments, the presently disclosed subject matter provides a method of preparing a targeted liposome. In some embodiments, the method comprises contacting an antibody or antibody fragment that binds to a cell surface antigen expressed by a malignant hematological cell population with a derivatized liposome. The derivatized liposome can be a liposome wherein the outer surface of the liposome includes (e.g., is attached to) a group that can form a covalent or non-covalent bond with a group on the antibody or antibody fragment. For instance, one of the lipid components of the liposome can be attached to a group that can be conjugated to (or modified to be conjugated to) an antibody, antibody fragment, or derivatized versions thereof. In some embodiments, the derivatized liposome is a derivatized version of a liposome that comprises a compound of Formula (I) (e.g., AD198).

In some embodiments, a polymer-modified lipid component of the presently disclosed liposomes contains a terminal group that can be used to conjugate the liposome to an antibody, antibody fragment, or derivatized version thereof. For example, PEG-functionalized lipids can be activated to contain various terminal reactive groups suitable for coupling to antibodies or antibody fragments by coupling methods known in the art. Generally, PEG chains are functionalized to contain reactive groups suitable for coupling with groups present on the antibody or antibody fragment, such as thiols, amino groups, aldehydes, or ketones (e.g., derived from mild oxidation of carbohydrate portions of an antibody). Suitable reactive groups include, but are not limited to, maleimide (e.g., for reaction with thiols), N-hydroxysuccinimide (NHS) or NHS-carbonated ester (e.g., for reaction with primary amines), hydrazide or hydrazine (e.g., for reaction with aldehydes or ketones), haloacetyl (e.g., iodoacetyl, which preferentially react with thiols) and dithiopyridine (e.g., for reaction with thiols).

Many PEG-functionalized lipids with reactive terminal groups are commercially available (e.g., from Avanti Polar Lipids, Alabaster, Ala., United States of America or Nanocs, Inc., New York, N.Y., United States of America). They can also be synthesized according to reactions known in the art. For example, preparation of a maleimide-terminated PEG-functionalized lipid can be accomplished by contacting a PEG bis(amine) (e.g., PEG₂₀₀₀ bis(amine)) with 2-nitrobenzene sulfonyl chloride to generate a mono-protected PEG bis(amine). The mono-protected PEG bis(amine) can be reacted with carbonyl diimidizole in triethylamine to form an urea. The urea can then be reacted with DSPE or another lipid in triethylamine to form a PEG-derivatized lipid protected with 2-nitrobenzyl sulfonyl chloride. The protecting group can be removed with acid to provide a PEG-derivatized lipid having a free amino group at the end of the PEG chain. Reaction with maleic anhydride followed by reaction with acetic anhydride provides the maleimide-terminated PEG-functionalized lipid (e.g., DSPE-PEG₂₀₀₀-maleimide).

Typically, the PEG-functionalized lipid can be already activated with the reactive terminal group when the PEG-functionalized lipid is incorporated into the liposome. Thus, the liposome itself need not be exposed to the activating reagents used to introduce the terminal PEG reactive functionality and there is no need to remove reagent contaminants from the liposomes. However, in some embodiments, the reactive group can be added after the liposome is formed. Adding the reactive group after the liposome is formed results in the activation reaction being confined to the outer, surface-accessible lipids, and thus, the reactive groups can be completely quenched prior to use of the liposome in therapy. In addition, adding the reactive group after formation of the liposome can be done when the reactive group is unstable in water.

More particularly, maleimides are widely used protein modifying reagents and are especially useful when the maleimide is one of two functional groups in a heterobifunctional cross-linking reagent. The reaction of maleimides with thiols involves Michael addition of the thiol to the carbon-carbon double bond. Reaction with amino groups can occur by the same mechanism, but at a slower rate. Thus, since thiol is the most reactive species with maleimide, particularly at neutral pH, the maleimide group can be used to target thiols selectively.

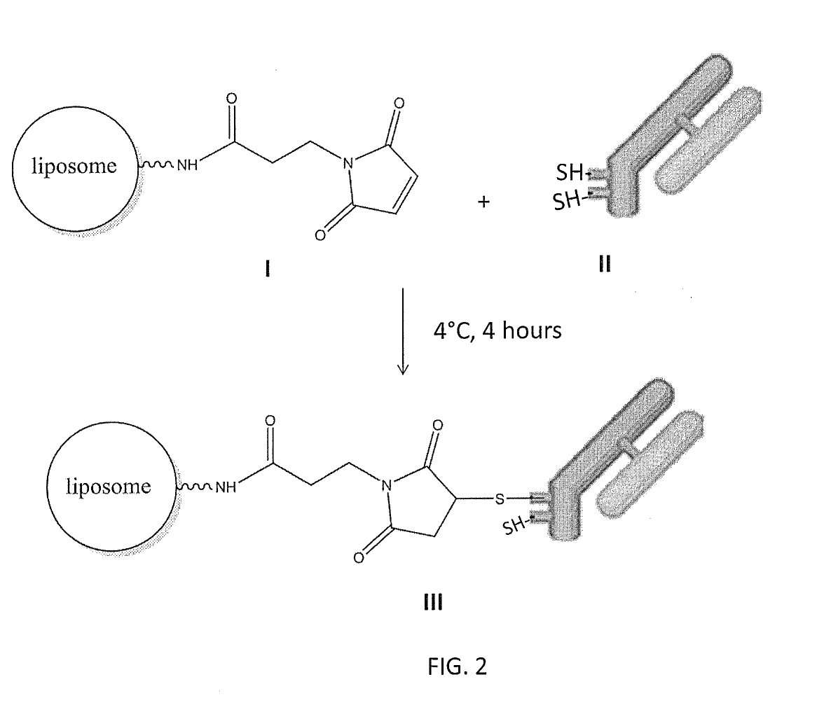

FIG. 2 shows a reaction scheme where maleimide-derivatized liposome I is contacted with Fab′ fragment II (which contains free thiol groups) for four hours at 4° C. to provide Fab′ derivatized liposome III. Fab′ fragment II can be prepared by reaction of a whole antibody with pepsin, which cleaves the antibody below the hinge region to provide an (Fab′)₂ fragment and fragments from the Fc region. The (Fab′)₂ fragment can be subjected to mild reduction (e.g., with dithiothreitol, DTT) to provide two Fab′ fragments. In place of Fab′ fragment II of FIG. 2, another type of antibody fragment or whole antibody that contains a free thiol group can be used.

If the targeting antibody of interest does not contain a free thiol group, conjugation with a maleimide-derivatized liposome can still be performed by using a suitable cross-linking reagent. For example, a free primary amino group of the antibody or antibody fragment can be reacted with an S-acyl-containing cross-linking reagent to form an S-acyl-derivatized antibody or antibody fragment. S-acyl-containing cross-linking reagents include, for example, N-succinimidyl-S-acetylthioacetate (SATA) and S-acetylmercaptosuccinic anhydride (SAMSA). Reaction of the S-actyl-derivatized antibody or antibody fragment with an S-acyl deblocking agent (e.g., hydroxylamine or carboxymethoxylamine (CMA)) and a maleimide-derivatized liposome or haloacetyl-derivatized liposome, either sequentially (i.e., deblocking agent followed by derivatized liposome) or simultaneously, can provide a conjugate of the antibody or antibody fragment and the liposome. In some embodiments, e.g., when CMA is the deblocking agent, the deblocking reagent can be used in combination with a chelating agent. Suitable chelating agents include, but are not limited to, ethylenediamine tetraacetic acid (EDTA) or diethylenetriamine pentaacetic acid (DTPA).

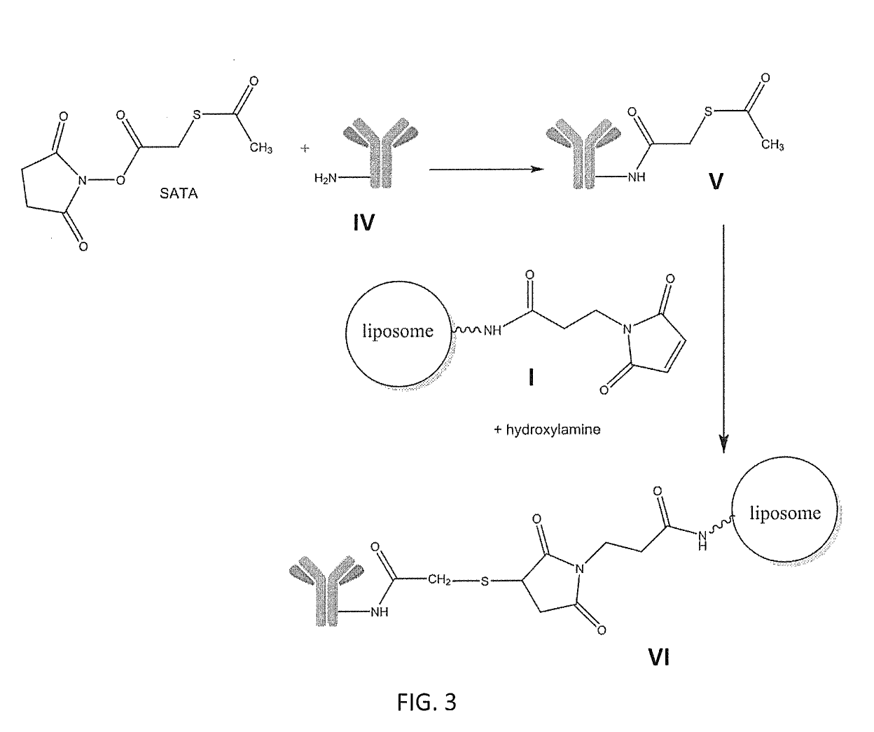

FIG. 3 shows a reaction scheme where an antibody without a free thiol group, i.e., antibody IV, is contacted with SATA (e.g., at pH 7 for 30 minutes at about 30° C.) to form S-acyl-derivatized antibody V and side product N-hydroxysuccinimide (NHS, not shown). S-acyl-derivatized antibody V can be purified (e.g., via gel filtration chromatography), as necessary. Prior to further reaction of the derivatized antibody, it can also be analyzed for protein and/or thiol concentration (e.g., via the Bradford method for protein and/or via Ellman's method for thiol). Then the S-acyl-derivatized antibody V is contacted with maleimide-derivatized liposome I and hydroxylamine (e.g., overnight at 4° C.) to form conjugate VI, which contains a thioether and, as a side product, acetohydroxamic acid. Conjugate VI can be purified (e.g., via ultrafiltration) and analyzed to assess conjugation. For example, Western blotting can be performed to determine changes to the size and weight of the liposome and antibody starting materials.

Preparation of the derivatizable liposomes themselves can be by any suitable method known in the art. Methods for preparation of liposomes include, but are not limited to, the lipid film hydration method, the ether injection method, the ethanol injection method, reverse evaporation method, and the detergent removal method.

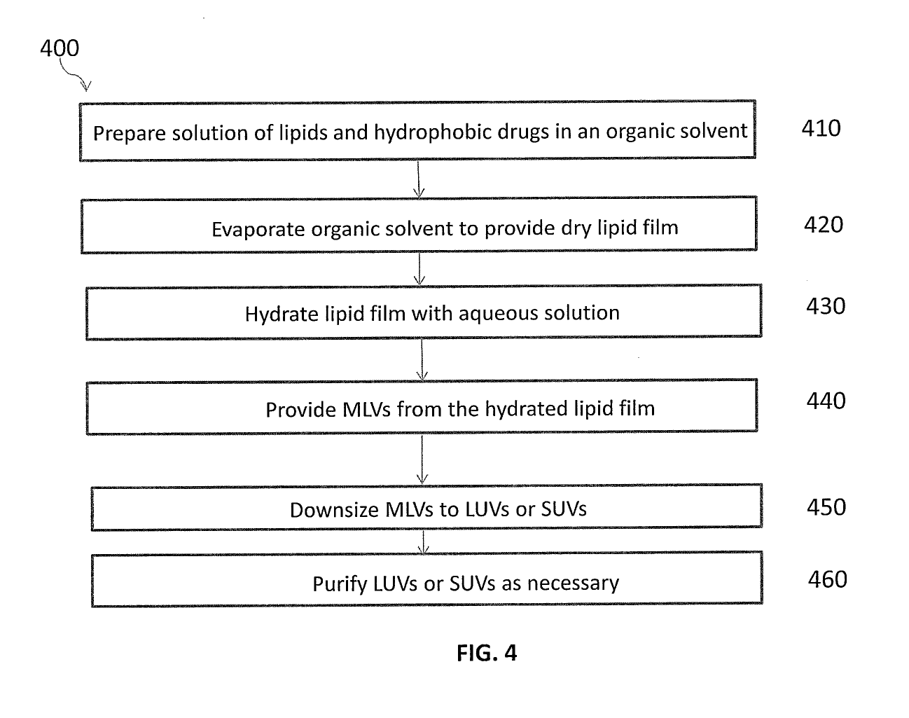

For example, in some embodiments, multilamellar vesicles (MLVs) can be prepared by a lipid film hydration method (i.e., the Bangham method) and downsized. FIG. 4 shows a flow chart of such a method. More particularly, FIG. 4 shows liposome preparation method 400, wherein in step 410, a solution of liposome forming lipids and a hydrophobic drug compound (i.e., a compound of Formula (I)) is prepared in a suitable organic solvent. Suitable organic solvents can include, but are not limited to, chloroform or mixtures of chloroform and methanol. In step 420, the organic solvent is evaporated (e.g., by using a rotary evaporator under reduced pressure or, if a small volume of solvent is to be evaporated, by using a gas steam) to provide a dry lipid film. The lipid film is hydrated in step 430. Hydration can comprise covering the lipid film with an aqueous medium and agitating the resulting mixture. The temperature of the aqueous medium is typically above the gel-liquid crystal transition temperature of the lipids. Upon spinning, stirring, and/or shaking the mixture of the lipid film and the aqueous solution in step 440 (e.g., spinning the mixture in a warm water bath of a rotary evaporator without applying vacuum), MLVs are formed. Alternatively, MLVs can be prepared by vortexing the lipid film in a buffered aqueous solution.

The MLVs can then be downsized in step 450. Any suitable method can be used for the downsizing, e.g., extrusion, sonication, homogenization, or microfluidization. In some embodiments, the downsizing of step 450 involves extruding the MLVs through a membrane (e.g., a polycarbonate membrane having a suitable pore size) or small orifice (i.e., the French pressure cell extrusion method). When membrane extrusion is used, the pore size of the membrane typically corresponds to about the largest diameter of the liposomes produced. The liposomes can be extruded two or more times through the same membrane. Downsizing can produce large unilamellar vesicles (LUVs, i.e., having a diameter above about 100 nm) and/or small unilamellar vesicles (SUVs, i.e., having a diameter between about 20 and 100 nm). In step 460, the LUVs and/or SUVs can be further purified, e.g., via centrifugation, dialysis, untrafiltration, and/or column chromatography. Following purification, the liposomes can be conjugated to antibodies or antibody fragments as described hereinabove. Alternatively, an antibody-lipid or antibody fragment-lipid can be formed and incorporated into a liposome during liposome preparation.

### EXAMPLES

The following Examples have been included to illustrate modes of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Optimization of Untargeted Liposomes

Initial optimization of the liposomes was performed using untargeted liposomes prepared using a lipid thin film hydration method as described above. More particularly, untargeted liposomes prepared from different mixtures of phospholipids and from different starting concentrations of lipid and AD198 were prepared to study the effects of these parameters on liposome size and drug loading. Following liposome preparation via the Bangham method, free (i.e., non-liposome-entrapped) drug was removed via dialysis and the liposome composition was analyzed for size, charge (i.e., zeta) potential, and drug loading. Liposome size was determined via transmission electron microscopy (TEM) or dynamic light scattering (DLS) techniques. Charge (zeta (C)) potential was analyzed using laser doppler electrophoresis. Drug loading was assessed via high performance liquid chromatograph (HPLC), using a λₘₐₓ for AD198 of 254 nm. Liposome dissolution was studied using micro-dialysis cassettes in phosphate buffered saline at 37° C.

Differential scanning calorimetry (DSC) was used to study the thermal behaviors of AD198, HSPC, EPC and various combinations thereof (e.g., binary mixtures of AD198 with 20%, 40%, 50%, 60%, or 80% by weight HSPC or EPC). The DSC parameters used were as follows: pan type was Tzero aluminum hermetic; start temperature was 10° C.; end temperature was 150° C.; ramp rate was 3° C. per minute; and the average sample weight was 5 mg. In the DSC profiles obtained, the glass transition temperature (T_{g}) of AD198 was observed at 20% EPC concentration. The T_{g} of AD198 was also observed at up to 40% HSPC concentration. The DSC data indicates which ratios of drug and lipid give complete fusion and can give a preliminary understanding of what ratios of AD198 and lipid can be utilized to produce drug-loaded liposomes.

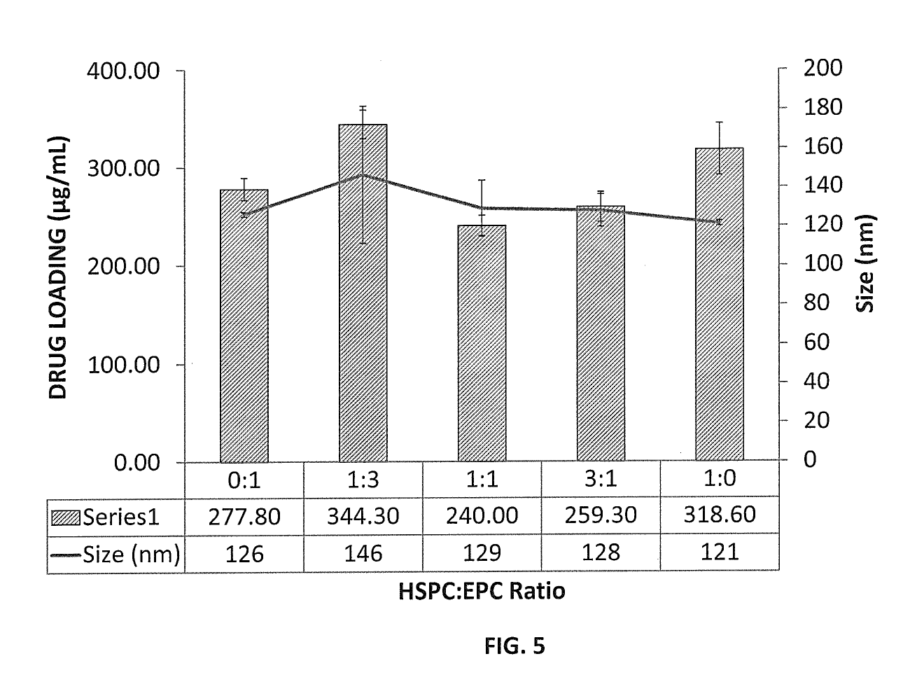

FIG. 5 shows the effects of using different ratios of HSPC to EPC on drug loading and liposome size in liposomes prepared using an initial concentration of AD198 of 1 mg/ml. The highest drug loading (344.30 μg/mL) was observed using a 1:3 ratio of HSPC to EPC. These liposomes were also slightly larger than liposomes prepared only from HSPC, only with EPC, with a 1:1 ratio of HSPC to EPC, or with a 3:1 ratio of HSPC to EPC. The liposomes prepared with only HSPC combined relatively high drug loading (318.60 μg/mL) and relatively small size (121 nm).

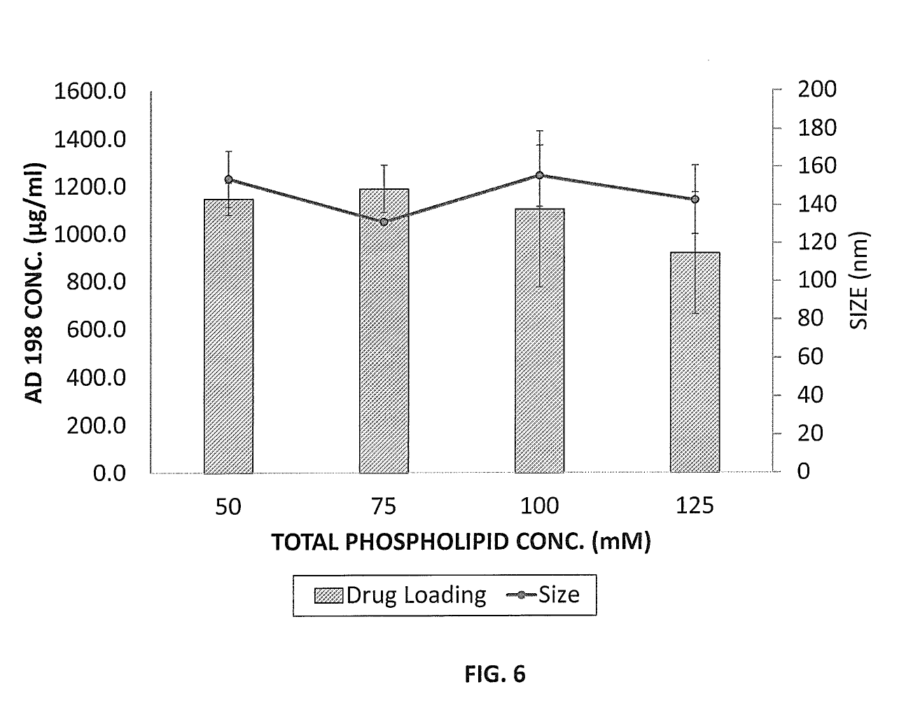

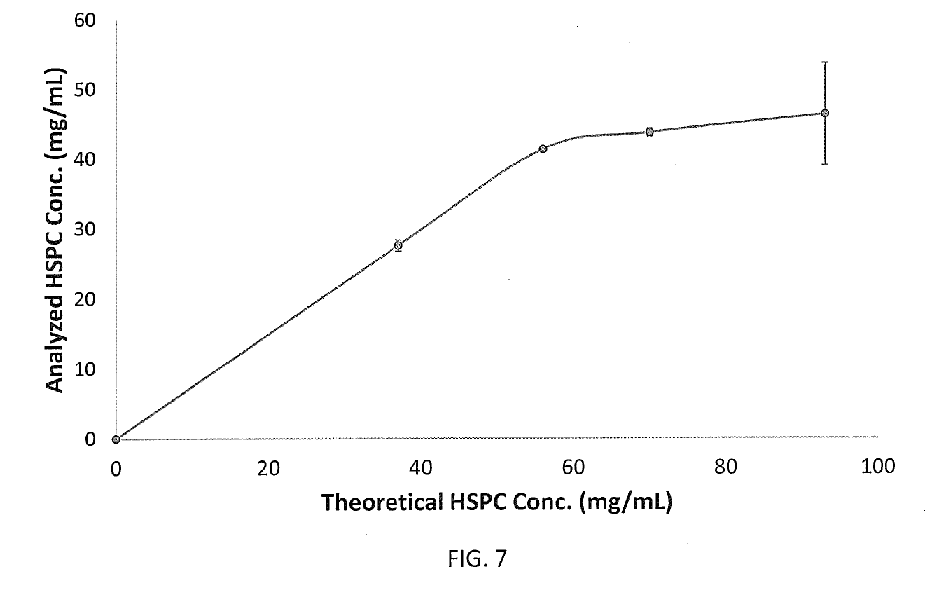

FIG. 6 shows the effect of total phospholipid concentration on drug loading and liposome size in liposomes prepared using an initial concentration of AD198 of 2 mg/ml and an initial concentration of phospholipid ranging from 50 mM to 125 mM. The liposomes prepared using a 75 mM total phospholipid concentration provided both high drug loading and relatively smaller size. FIG. 7 shows that the concentration of phospholipid present in prepared liposomes increases with the concentration of phospholipid used to prepare the liposomes until the concentration of phospholipid used to prepare the liposomes is about 60 mg/mL. Above that concentration, the amount of phospholipid incorporated into the liposomes appears increases to a lesser extent as initial phospholipid concentration increases.

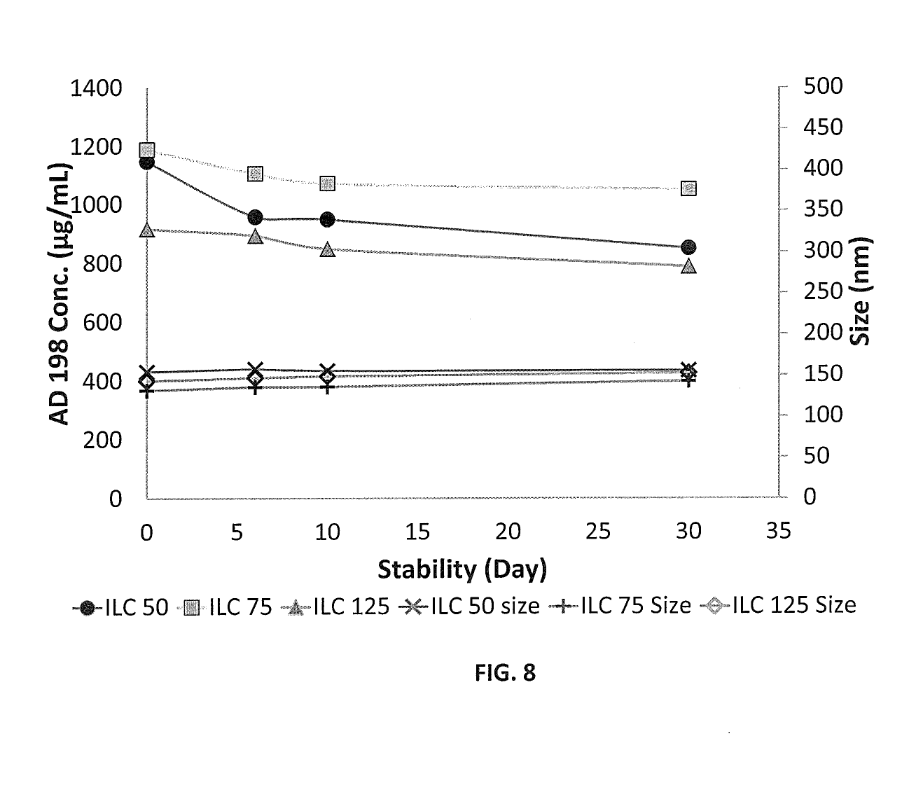

FIG. 8 shows liposomal stability at about 5° C. (±3° C.). In liposomes prepared using an initial phospholipid concentration of 50 mM (ILC 50), 75 mM (ILC 75) or 125 mM (ILC 125), liposome size remains stable for up to 30 days.

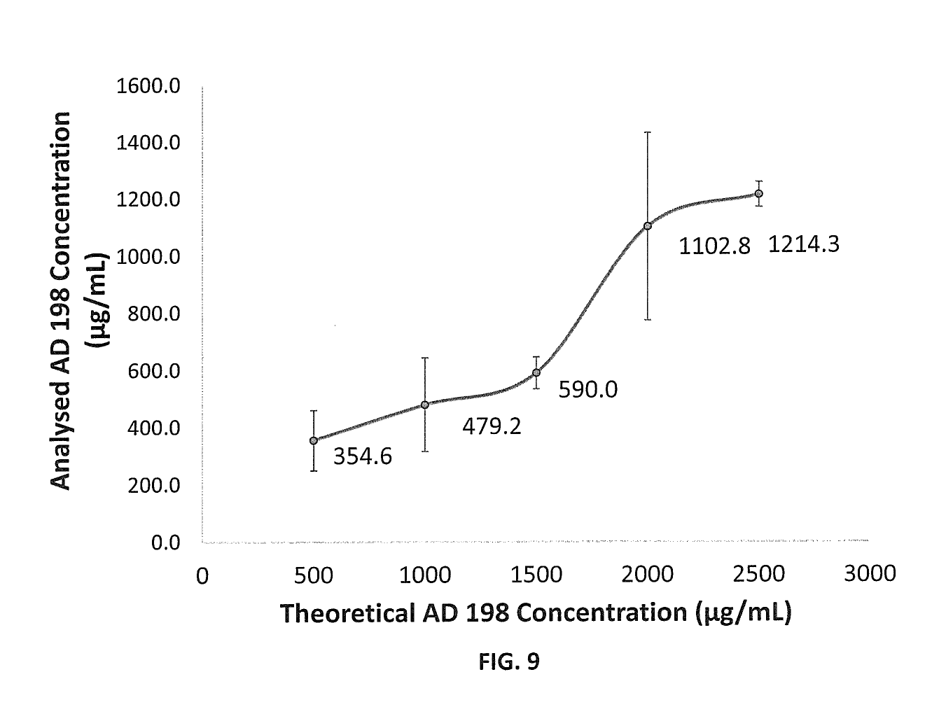

The liposomes also retained about 80% or more of their initial AD198 concentration over 30 days at about 5° C. Drug retention was higher in liposomes prepared with the higher phospholipid concentrations (i.e., ILC 75 and ICL 125), than in liposomes prepared from the lower phospholipid concentration (i.e., ILC 50). FIG. 9 shows the dependence of drug loading on drug concentration during liposomal preparation.

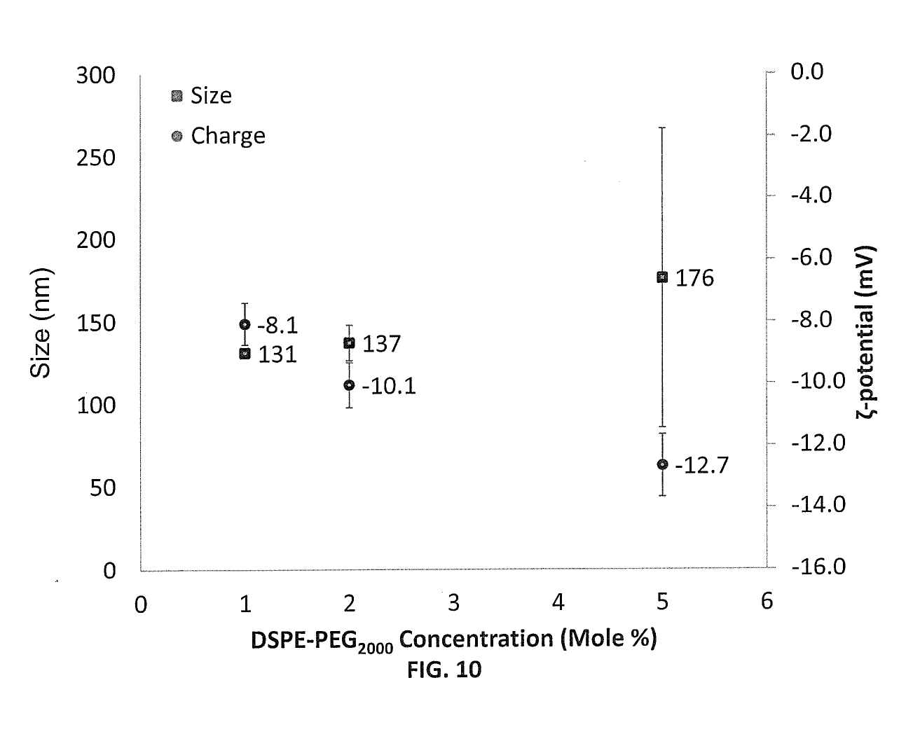

FIG. 10 shows the dependence of liposomal surface charge and size on the concentration of DSPE-PEG₂₀₀₀ used during liposome preparation (i.e., 1 mole %, 2 mole %, or 5 mole %). In particular, the liposomes prepared using the two lower levels of DSPE-PEG₂₀₀₀ exhibited both good surface charge and size characteristics.

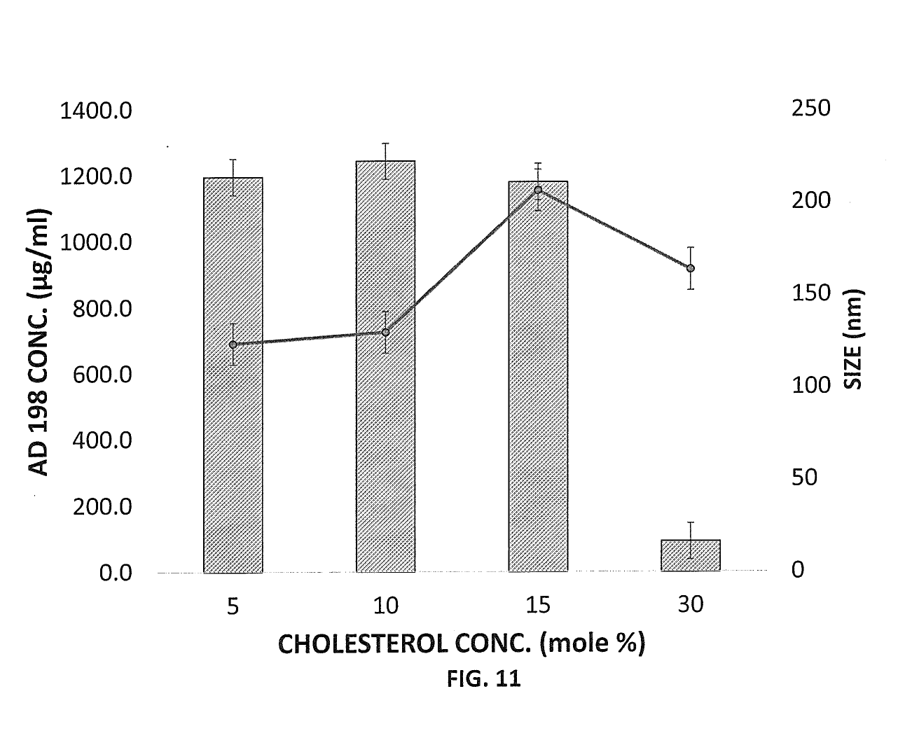

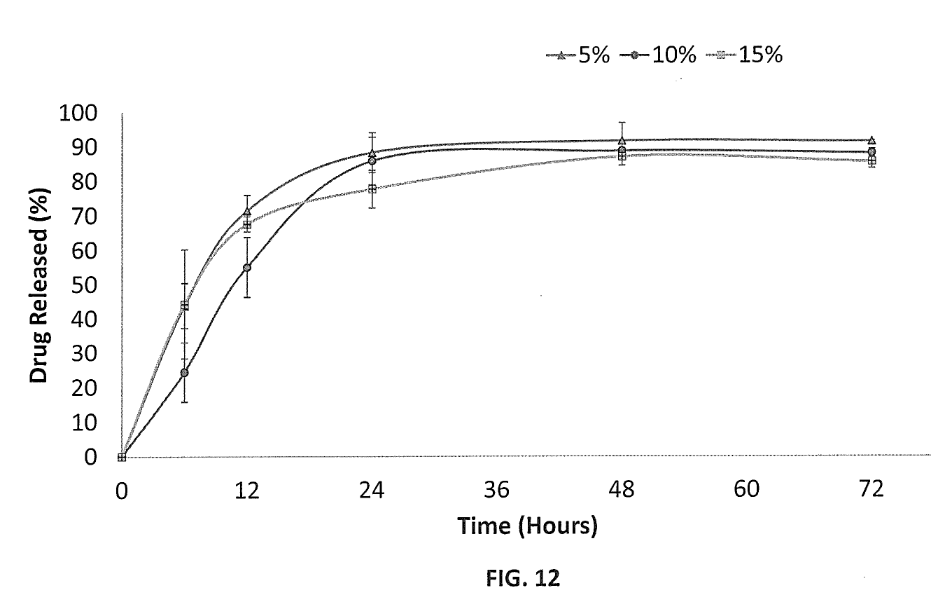

FIG. 11 is a graph showing how the amount of cholesterol used to prepare the liposomes affects liposomal drug loading and size. Drug loading and size were both in preferred ranges in liposomes prepared using 5 mole % and 10 mole % cholesterol. At 15 mole % cholesterol, drug loading was still good, but liposomal size was larger. The use of 30 mole % cholesterol resulted in a reduction in drug loading. FIG. 12 shows how cholesterol affects liposomal dissolution at physiologically relevant temperatures. More particularly, at 37° C., at least about 50% of the liposomal composition dissolved within 12 hours in liposomes prepared using 5, 10, or 15 mole % cholesterol. Within 24 hours between about 75% and about 85% of the liposomal compositions had dissolved.

### Example 2

### Preparation of Targeted Liposomes

Targeted AD-198-loaded liposomes were prepared as outlined in FIG. 3 by contacting SATA with an anti-CD22 monoclonal antibody (Pierce catalog number MA1-7635, Pierce Antibodies, Thermo Fisher Scientific, Rockford, Ill., United States of America). More particularly, the targeted liposomes were prepared by mixing 100 microliters (μl) of SATA solution (10 mM SATA in DMF) with 900 μl of anti-CD22 solution (0.1 μM anti-CD22 antibody in PBS). The mixture was gently stirred at room temperature for 30 minutes. After 30 minutes, free SATA was separated from the SATA-derivatized antibody using gel chromatography using a PD-10 column.

A 1 ml aliquot of liposomes containing 2 mole % of maleimide derivatized lipid was added to the derivatized antibody. Along with the liposomes was added deacylation buffer at a ratio of derivatized antibody solution to deacylation buffer of 10:1 (i.e., for 1 ml of purified derivatized antibody, 100 μl of deacylation buffer was added). The deacylation buffer contained 0.5 M hydroxylamine hydrochloride and 25 mM EDTA, disodium salt in PBS (pH=7.4, adjusted using NaOH). The liposome/derivatized antibody/deacylation buffer mixture was incubated for 12 hours at 4° C. with gentle shaking. Afterward, the antibody conjugated liposomes were separated from unconjugated antibody by ultrafiltration using a 200,000 molecular weight cut-off (MWCO) filter. Liposome dispersions were stored at 4° C.

### Example 3

### Cytotoxicity of Targeted Liposomes

Fluorescently tagged, targeted, AD198-loaded liposomes were prepared in a manner similar to that described in Example 2. These liposomes were incubated with either CD22+(Daudi) or CD22− (Jurkat) cells. For comparison, fluorescently tagged, untargeted, AD198-loaded liposomes were also incubated with the cells. After 30 minutes, cells were analyzed for liposomal and drug uptake via confocal laser scanning microscopy. Analysis indicated significant cellular uptake of both targeted liposome and AD198 in Daudi cells. Co-localization of AD198 and liposomes in the cells, as well as the time-scale of cellular uptake, indicated intact liposomal uptake into cells, e.g. via endocytosis. The exact mechanism of endocytosis can be determined by comparing cellular uptake with and without pretreatment of cells with different inhibitors (e.g., filipin III, a inhibitor of caveolae-mediated endocytosis; or chlorpromazine, an inhibitor of clathrin-mediated endocytosis).

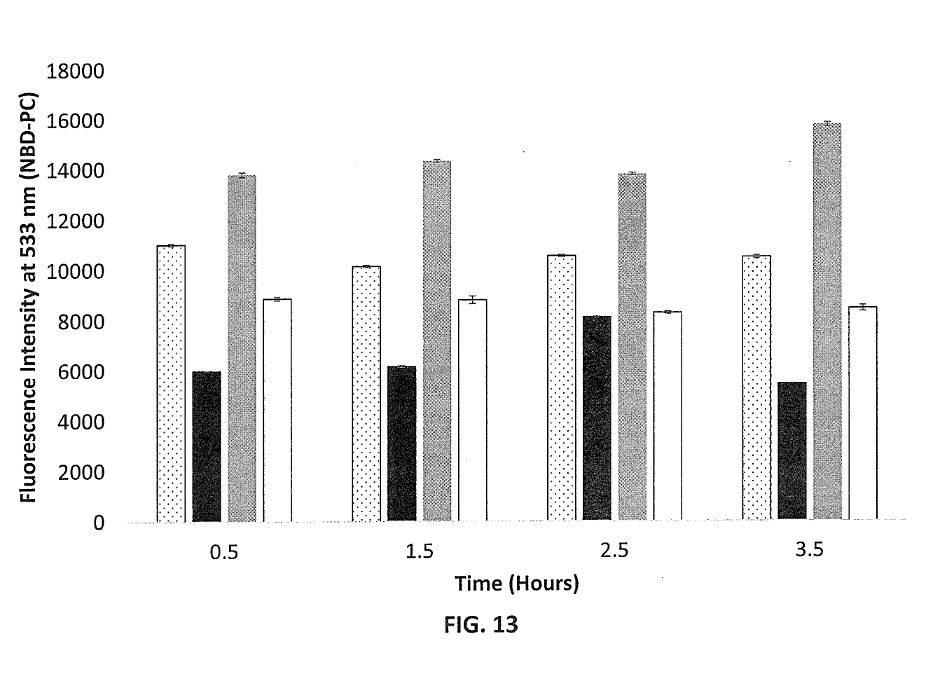

Cellular uptake of liposomal drug was also studied via microscopy at different time points for up to 3.5 hours. It appeared that both targeted and untargeted drug could enter either type of cell (i.e., Daudi or Jurkat cell). However, CD22 targeted drug entered Daudi cells at a significantly higher concentration compared to untargeted liposomal drug in both the Daudi and Jurkat cells. Average fluorescence intensity due to uptake of the liposomes is shown in FIG. 13.

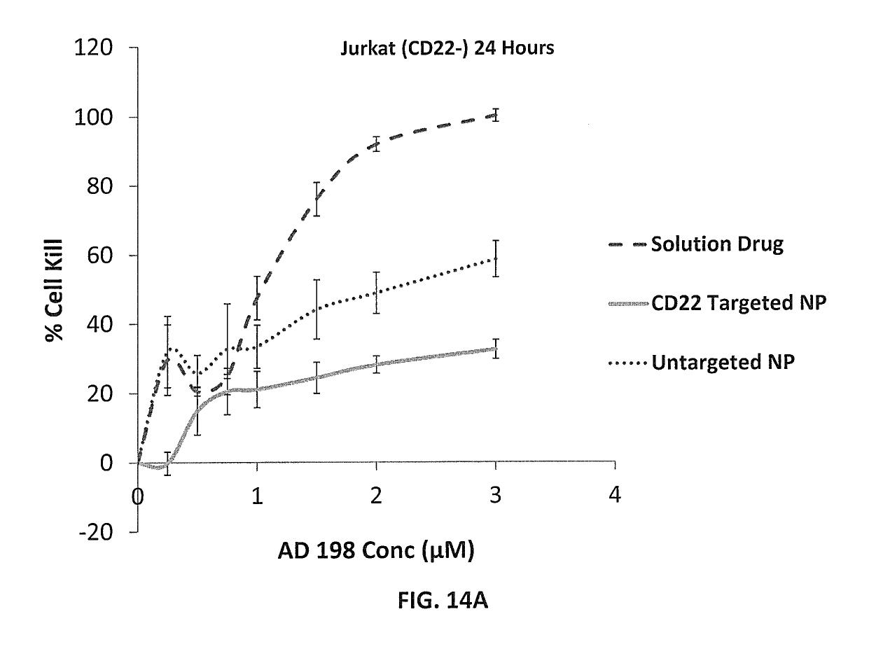

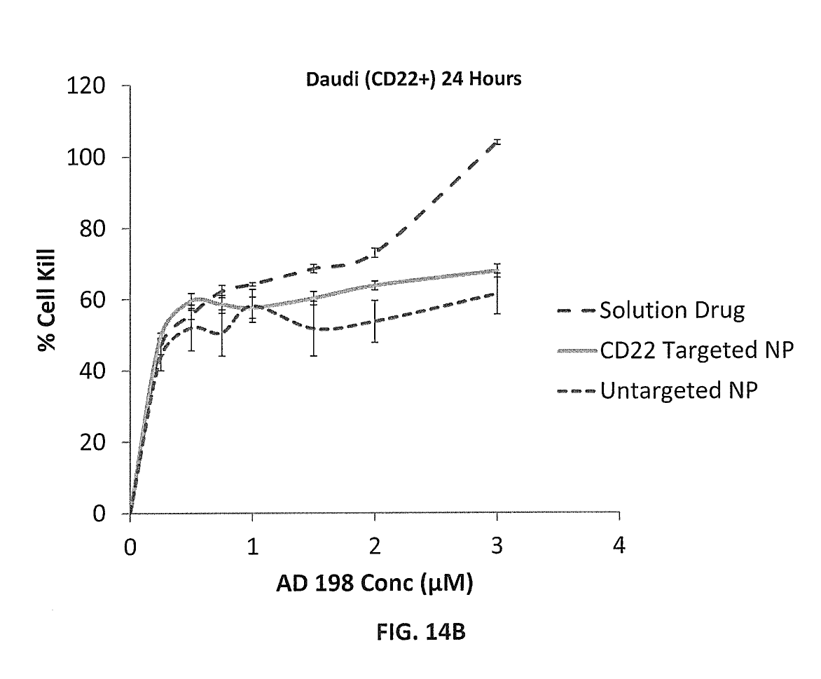

In vitro efficacy of the liposomes was also studied using the MTT assay to assess cell viability. Daudi or Jurkat cells were incubated with different concentrations of targeted, drug loaded liposomes; with untargeted, drug-loaded liposomes, or (as a positive control) with free drug in solution (i.e., “solution drug”) for 24 hours. The percentages of cells killed (% cell killed) by the liposomes or the solution drug are shown in FIGS. 14A and 14B. IC₅₀ concentrations were also calculated for the solution drug and the targeted liposome. More particularly, in Jurkat cells, the IC₅₀ of free AD198 was 1.05 μM, while the IC₅₀ for the targeted liposomes was greater than 3.0 μM. In Daudi cells, the IC₅₀s of the free AD198 and the targeted liposomes was about the same, i.e., 0.25 μM. The higher sensitivity of the CD22+ cells to the targeted liposomes (e.g., the higher cytotoxicity of targeted liposomes in CD22+ cells as compared to CD22− cells) can provide a therapeutic advantage for the targeted liposomes in treating malignant B cells.

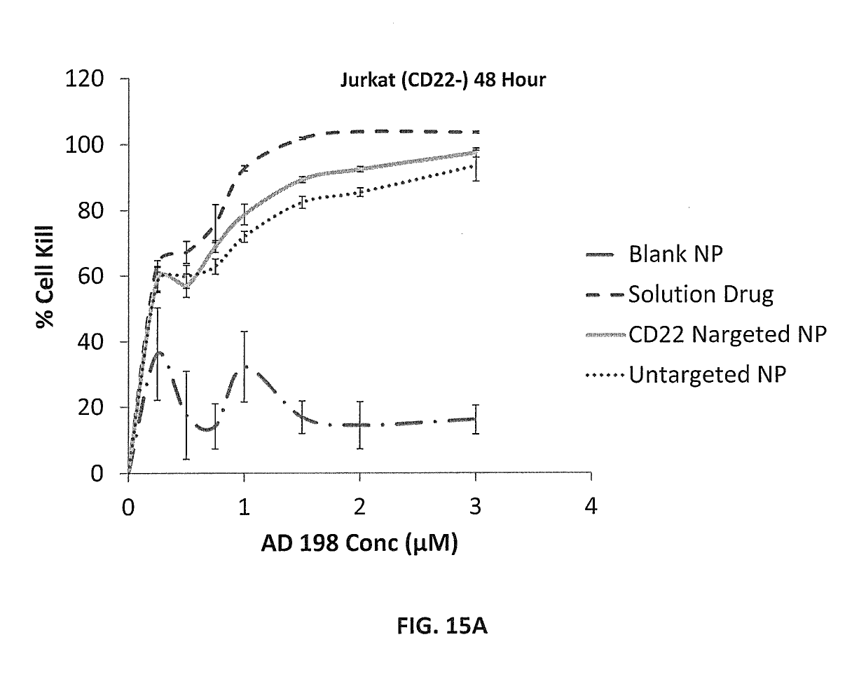

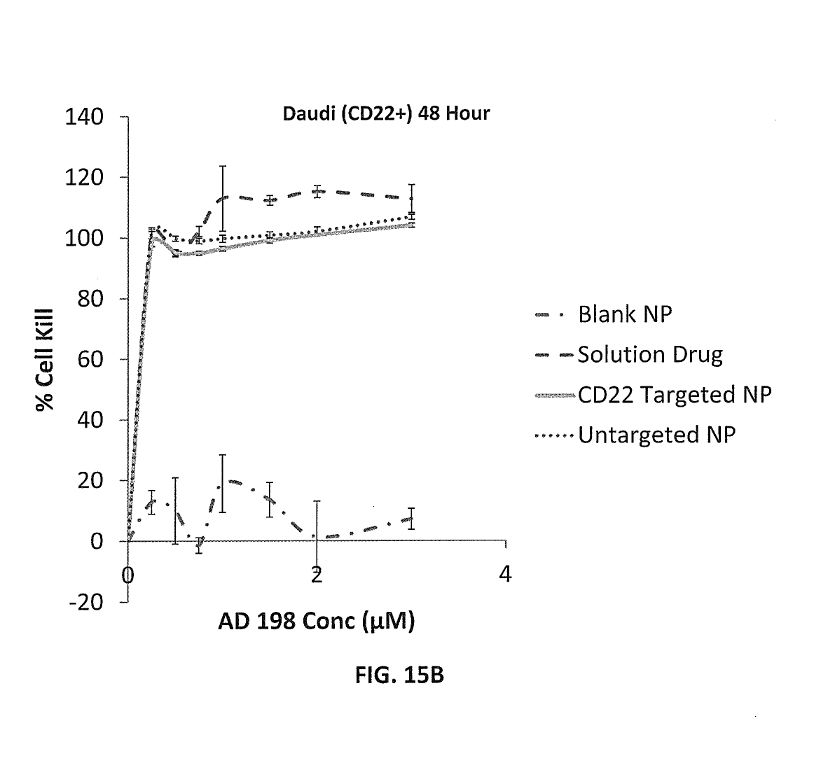

Daudi or Jurkat cells were also incubated with different concentrations of targeted, drug-loaded liposomes; with untargeted, drug-loaded liposomes; with free (solution) AD198; or with untargeted, drug-free liposomes at different concentrations for 48 hours. FIGS. 15A and 15B show the % cells killed after 48 hours. The IC₅₀ values for both free drug and targeted liposome in either the Duadi or the Jurkat cells was 0.1 μM.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating a subject having a disorder associated with a malignant hematological cell population, the method comprising administering to the subject an effective amount of a liposomal composition comprising a targeted liposome, wherein the targeted liposome comprises: (a) an outer liposomal surface attached to an antibody or antibody fragment that binds to a cell surface antigen expressed by the malignant hematological cell population, and (b) a compound of Formula (I): 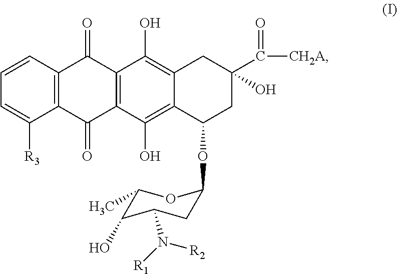
wherein:
A is a C₄-C₁₀ alkanoate moiety, hemiadipate, or hemiglutarate;
R₁ is H, n-propyl, or n-butyl;
R₂ is benzyl, n-propyl, or n-butyl; and
R₃ is H or methoxy;
or a pharmaceutically acceptable salt thereof.

2. The method of claim 1, wherein R₁ is H and R₂ is benzyl.

3. The method of claim 1, wherein A is a C₄-C₈ alkanoate moiety.

4. The method of claim 3, wherein A is selected from the group consisting of:

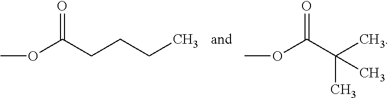

5. The method of claim 1, wherein the compound of Formula (I) is selected from the group consisting of:

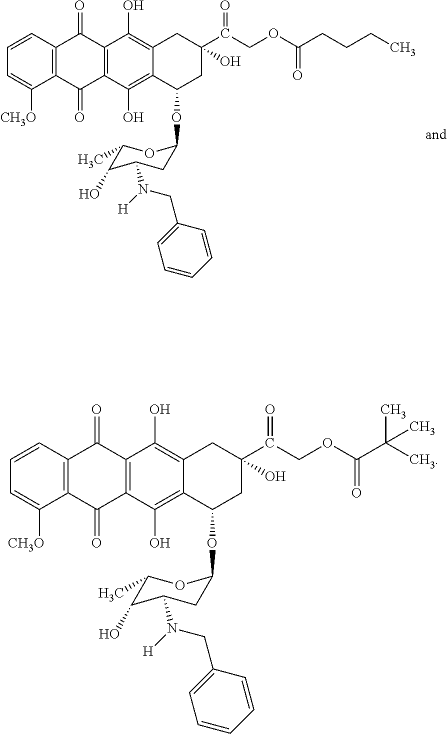

6. The method of claim 1, wherein the targeted antibody is a B cell- or a T cell-targeted antibody and the antibody or antibody fragment binds to a cell surface antigen expressed by a malignant B cell or a malignant T cell.

7. The method of claim 1, wherein the antibody or antibody fragment is an anti-CD3, anti-CD4, anti-CD5, anti-CD7, anti-CD8, anti-CD10, anti-CD13, anti-CD15, anti-CD19, anti-CD20, anti-CD21, anti-CD25, anti-CD74, or anti-CD22 antibody or fragment thereof.

8. The method of claim 7, wherein the antibody or antibody fragment is an anti-CD22 antibody or fragment thereof.

9. The method of claim 1, wherein the antibody or antibody fragment is a monoclonal antibody, a chimeric antibody, a humanized antibody, or a fragment thereof.

10. The method of claim 1, wherein the disorder is selected from the group consisting of a lymphoma, a leukemia, multiple myeloma, Waldenstrom's macroglobulinemia, and primary amyloidosis.

11. The method of claim 1, wherein the subject is a mammal.

12. The method of claim 1, wherein the outer liposomal surface is covalently attached to the antibody or antibody fragment via a bivalent linkage moiety.

13. The method of claim 12, wherein the bivalent linkage moiety includes a thioether.

14. The method of claim 12, wherein the antibody or antibody fragment is free of a free thiol moiety.

15. The method of claim 12, wherein the bivalent linkage moiety is attached to the antibody or antibody fragment via a covalent bond to an antibody or antibody fragment amino group.

16. The method of claim 1, wherein the liposomal composition further comprises a polyethylene glycol chain associated with an outer liposomal surface of the targeted liposome.

17. The method of claim 1, wherein the targeted liposome has a mean particle size between about 80 and about 200 nm; and/or a surface charge potential of between about −5 mV and about −40 mV; and/or a drug loading of between about 1 and about 100 micrograms per milligram (μg/mg) of lipid.

18. The method of claim 1, wherein the targeted liposome has a mean particle size between about 120 and about 140 nm.

19. The method of claim 1, wherein the targeted liposome has a surface charge potential of between about −8 and about −12 milliVolts (mV).

20. The method of claim 1, wherein the targeted liposome has a drug loading of about 20 μg of compound of Formula (I) to mg of lipid.

21. The method of claim 1, wherein the targeted liposome has a circulation half-life of about 24 hours.

22. The method of claim 1, wherein the targeted liposome is prepared from a lipid formulation comprising hydrogenated soybean phosphatidylcholine (HSPC) and egg phosphatidylcholine (EPC).

23. The method of claim 22, wherein the lipid formulation further comprises cholesterol, distearyl phosphatidylethanolamine-polyethylene glycol (DSPE-PEG), and distearyl phosphatidylethanolamine-polyethylene glycol-maleimide (DSPE-PEG-maleimide).

24. A liposomal composition comprising a targeted liposome, wherein the targeted liposome comprises: (a) an outer liposomal surface attached to an antibody or antibody fragment that binds to a cell surface antigen expressed by a malignant hematological cell population, and (b) a compound of Formula (I): 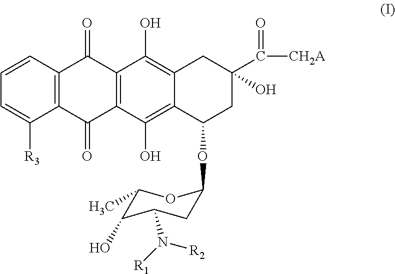
wherein:
A is a C₄-C₁₀ alkanoate moiety, hemiadipate, or hemiglutarate;
R₁ is H, n-propyl, or n-butyl;
R₂ is benzyl, n-propyl, or n-butyl; and
R₃ is H or methoxy;
or a pharmaceutically acceptable salt thereof.

25. The composition of claim 24, wherein R₁ is H and R₂ is benzyl.

26. The composition of claim 24, wherein A comprises a C₄-C₈ alkanoate.

27. The composition of claim 26, wherein A is selected from the group comprising:

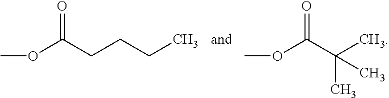

28. The composition of claim 24, wherein the compound of Formula (I) is selected from the group comprising:

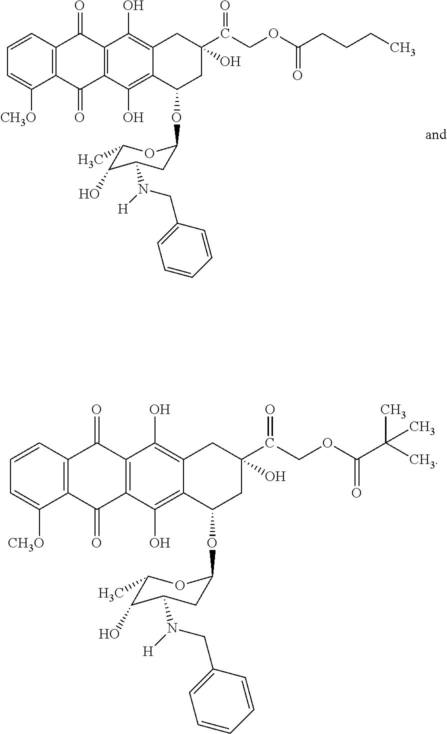

29. The composition of claim 24, wherein the targeted liposome is a B cell- or a T cell-targeted antibody and the antibody or antibody fragment binds to a cell surface antigen expressed by a malignant B cell or a malignant T cell.

30. The composition of claim 29, comprising a B cell-targeted liposome, wherein the B cell-targeted liposome is a liposome having an outer surface attached to an anti-CD22 antibody or antibody fragment.

31. The composition of claim 30, wherein the liposome is covalently attached to the anti-CD22 antibody or antibody fragment via a bivalent linkage moiety.

32. The composition of claim 31, wherein the bivalent linkage moiety comprises a thioether.

33. The composition of claim 31, wherein the bivalent linkage moiety is attached to the antibody or antibody fragment via a bond to an antibody or antibody fragment amino group.

34. The composition of claim 24, wherein the liposome further comprises a polyethylene glycol chain associated with an outer liposomal surface of the targeted liposome.

35. The composition of claim 24, wherein the targeted liposome has a mean particle size between about 80 and about 200 nm; and/or a surface charge potential of between about −5 mV and about −40 mV; and/or a drug loading of between about 1 and about 100 micrograms per milligram (μg/mg) of lipid.

36. The composition of claim 24, wherein the targeted liposome has a meant particle size between about 120 nm and about 140 nm.

37. The composition of claim 24, wherein the targeted liposome has a surface charge potential of between about −8 mV and about −12 mV.

38. The composition of claim 24, wherein the targeted liposome has drug loading of about 20 μg of the compound of Formula (I) per mg lipid.

39. The composition of claim 24, wherein the targeted liposome has a circulation half-life of about 24 hours.

40. The composition of claim 24, wherein the targeted liposome is prepared from a lipid formulation comprising hydrogenated soybean phosphatidylcholine (HSPC) and egg phosphatidylcholine (EPC).

41. The composition of claim 40, wherein the lipid formulation further comprises cholesterol, distearyl phosphatidylethanolamine-polyethylene glycol (DSPE-PEG), and distearyl phosphatidylethanolamine-polyethylene glycol-maleimide (DSPE-PEG-maleimide).

42. A method of preparing a targeted liposome, the method comprising contacting an antibody or antibody fragment that binds to a cell surface antigen expressed by a malignant hematological cell population with a derivatized liposome, wherein the derivatized liposome has an outer liposomal surface derivatized with a group that can form a bond with a group on the antibody or antibody fragment.

43. The method of claim 42, wherein the outer liposomal surface group is a maleimide group or a haloacetyl group.

44. The method of claim 42, further comprising contacting the antibody or antibody fragment with an S-acyl-containing cross-linking reagent to provide an S-acyl-derivatized antibody or antibody fragment, and contacting the S-acyl-derivatized antibody or antibody fragment with a S-acyl deblocking reagent to provide an antibody or antibody fragment having a free thiol group; prior to contacting the antibody or antibody fragment with the derivatized liposome.

45. The method of claim 44, wherein the S-acyl-containing cross-linking reagent is N-succinimidyl-S-acetylthioacetate (SATA) or S-acetylmercaptosuccinic anhydride (SAMSA).

46. The method of claim 44, wherein the antibody or antibody fragment is free of a free thiol moiety prior to the contacting steps.

47. The method of claim 44, wherein the deblocking agent is hydroxylamine or carboxymethoxylamine.

48. The method of claim 44, wherein the deblocking agent is contacted to the S-acyl-derivatized antibody or antibody fragment in combination with a chelating agent.

49. The method of claim 48, wherein the chelating agent is ethylenediamine tetraacetic acid (EDTA) or diethylenetriamine pentaacetic acid (DTPA).

50. The method of claim 42, wherein the antibody or antibody fragment is a monoclonal antibody, a chimeric antibody, a humanized antibody, or a fragment thereof.

51. The method of claim 42, wherein the antibody of antibody fragment binds to a surface antigen expressed by a malignant B cell or T cell.

52. The method of claim 42, wherein the antibody or antibody fragment is an anti-CD3, anti-CD4, anti-CD5, anti-CD7, anti-CD8, anti-CD10, anti-CD13, anti-CD15, anti-CD19, anti-CD20, anti-CD21, anti-CD25, anti-CD74 or anti-CD22 antibody or fragment thereof.

53. The method of claim 52, wherein the antibody or antibody fragment is an anti-CD22 antibody or fragment thereof.

54. The method of claim 42, wherein the derivatized liposome comprises a compound of Formula (I): 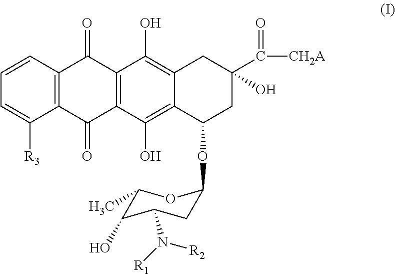
wherein:
A is a C₄-C₁₀ alkanoate moiety, hemiadipate, or hemiglutarate;
R₁ is H, n-propyl, or n-butyl;
R₂ is benzyl, n-propyl, or n-butyl; and
R₃ is H or methoxy;
or a pharmaceutically acceptable salt thereof.
